(19) United States
(12) Patent Application Publication
(10) Pub. No.: US 2011/0172232 A1
(43) Pub. Date: Jul. 14, 2011

(54) AMINOACYL PRODRUGS AS AN ACTIVE PHARMACEUTICAL INGREDIENT FOR THROMBOEMBOLIC DISORDERS

(75) Inventors: Hans-Georg Lerchen, Leverkusen (DE); Ursula Krenz, Leichlingen (DE); Michael Härter, Leverkusen (DE); Mark Jean Gnoth, Mettmann (DE); Georges Von Degenfeld, Leverkusen (DE); Elke Dittrich-Wengenroth, Wuppertal (DE); Anja Buchmüller, Essen (DE); Susanne Röhrig, Hilden (DE); Swen Allerheiligen, Essen (DE); Elisabeth Perzborn, Wuppertal (DE); Christoph Gerdes, Koln (DE); Karl-Heinz Schlemmer, Wuppertal (DE); Metin Akbaba, Ratingen (DE)

(73) Assignee: Bayers Schering Pharma Aktiengesellschaft, Berlin (DE)

(21) Appl. No.: 12/668,590

(22) PCT Filed: Jun. 28, 2008
(86) PCT No.: PCT/EP2008/005303

§ 371 Date: Jun. 21, 2010

(30) Foreign Application Priority Data
Jul. 11, 2007 (DE) 10 2007 032 345.1

(51) Int. Cl. A61K 31/5377; C07D 413/14; A61P 7/02

(52) U.S. Cl. 514/236.8; 544/137

(57) ABSTRACT

The present application relates to prodrug derivatives of 5-chloro-N-({(5S)-3-[2-fluoro-4-(3-oxomorpholin-4-yl)phenyl]-2-oxo-1,3-oxazolidin-5-yl}methyl)thiophene-2-carboxamide, processes for their preparation, their use for the treatment and/or prophylaxis of diseases, and their use for the manufacture of medicaments for the treatment and/or prophylaxis of diseases, especially of thromboembolic disorders.

The present application relates to prodrug derivatives of 5-chloro-N-({(5S)-3-[2-fluoro-4-(3-oxomorpholin-4-yl)phenyl]-2-oxo-1,3-oxazolidin-5-yl}methyl)thiophene-2-carboxamide, processes for their preparation, their use for the treatment and/or prophylaxis of diseases, and their use for the manufacture of medicaments for the treatment and/or prophylaxis of diseases, especially of thromboembolic disorders.

Prodrugs are derivatives of an active ingredient which undergo in vivo an enzymatic and/or chemical biotransformation in one or more stages before the actual active ingredient is liberated. A prodrug residue is ordinarily used in order to improve the profile of properties of the underlying active ingredient [P. Ettmayer et al., J. Med. Chem. 47, 2393 (2004)]. In order to achieve an optimal profile of effects it is necessary in this connection for the design of the prodrug residue as well as the desired mechanism of liberation to be coordinated very accurately with the individual active ingredient, the indication, the site of action and the administration route. A large number of medicaments is administered as prodrugs which exhibit an improved bioavailability by comparison with the underlying active ingredient, for example achieved by improving the physicochemical profile, specifically the solubility, the active or passive absorption properties or the tissue-specific distribution. An example which may be mentioned from the wide-ranging literature on prodrugs is: H. Bundgaard (Ed.), Design of Prodrugs: Bioreversible derivatives for various functional groups and chemical entities, Elsevier Science Publishers B.V., 1985.

5-Chloro-N-({(5S)-3-[2-fluoro-4-(3-oxomorpholin-4-yl)phenyl]-2-oxo-1,3-oxazolidin-5-yl}methyl)thiophene-2-carboxamide [compound (A)] is an orally effective, direct inhibitor of the serine protease factor Xa which performs an essential function in regulating the coagulation of blood. An oxazolidinone compound is currently undergoing in-depth clinical examination as a possible new active pharmaceutical ingredient for the prevention and therapy of thromboembolic disorders [S. Roehrig et al., J. Med. Chem. 48, 5900 (2005)].

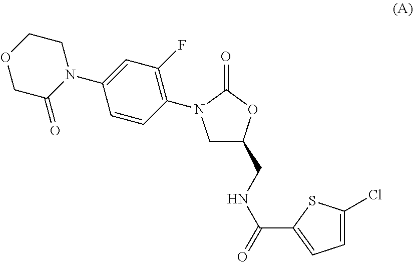

However, compound (A) has only a limited solubility in water and physiological media, making for example intravenous administration of the active ingredient difficult. It was therefore an object of the present invention to identify derivatives or prodrugs of compound (A) which have an improved solubility in the media mentioned and, at the same time, allow controlled liberation of the active ingredient (A) in the patient's body after administration.

WO 2005/028473 describes acyloxymethylcarbamate prodrugs of oxazolidinones which serve to increase the oral bioavailability. WO 01/00622 discloses acyl prodrugs of carbamate inhibitors of inosine-5′-monophosphate dehydrogenase. A further type of amide prodrugs for oxazolidinones which liberate the underlying active ingredient by a multistage activation mechanism is described in WO 03/006440.

The present invention relates to compounds of the general formula (I)

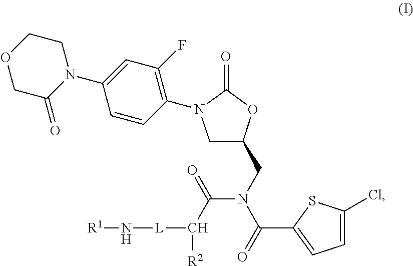

in which

R¹ is hydrogen or (C₁-C₄)-alkyl which may be substituted by hydroxy or (C₁-C₄)-alkoxy,

R² is hydrogen or (C₁-C₄)-alkyl,

and

L is a (C₁-C₄)-alkanediyl group in which one CH₂ group may be replaced by an O atom, or is a group of the formula

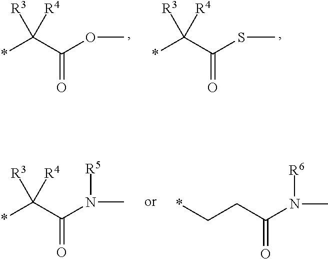

in which

* means the point of linkage to the N atom,

R³ is the side group of a natural α-amino acid or its homologs or isomers,

or

R³ is linked to R¹ and the two together form a (CH₂)₃ or (CH₂)₄ group,

R⁴ is hydrogen or methyl,

R⁵ is (C₁-C₄)-alkyl,

and

R⁶ is hydrogen or (C₁-C₄)-alkyl,

and the salts, solvates and solvates of the salts thereof.

Compounds according to the invention are the compounds of the formula (I) and the salts, solvates and solvates of the salts thereof, the compounds which are encompassed by formula (I) and are of the formulae mentioned hereinafter, and the salts, solvates and solvates of the salts thereof, and the compounds which are encompassed by formula (I) and are mentioned hereinafter as exemplary embodiments, and the salts, solvates and solvates of the salts thereof, insofar as the compounds encompassed by formula (I) and mentioned hereinafter are not already salts, solvates and solvates of the salts.

The compounds according to the invention may, depending on their structure, exist in stereoisomeric forms (enantiomers, diastereomers). The invention therefore relates to the enantiomers or diastereomers and respective mixtures thereof. The stereoisomerically pure constituents can be isolated in a known manner from such mixtures of enantiomers and/or diastereomers.

Where the compounds according to the invention can occur in tautomeric forms, the present invention encompasses all tautomeric forms.

Salts preferred for the purposes of the present invention are physiologically acceptable salts of the compounds according to the invention. However, salts which are themselves unsuitable for pharmaceutical applications but can be used for example for isolating or purifying the compounds according to the invention are also encompassed.

Physiologically acceptable salts of the compounds according to the invention include acid addition salts of mineral acids, carboxylic acids and sulfonic acids, e.g. salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid.

Solvates refer for the purposes of the invention to those forms of the compounds according to the invention which form a complex in the solid or liquid state through coordination with solvent molecules. Hydrates are a specific form of solvates in which the coordination takes place with water. Solvates preferred in the context of the present invention are hydrates.

In the context of the present invention, the substituents have the following meaning unless otherwise specified:

(C₁-C₄)-Alkyl and (C₁-C₃)-alkyl are in the context of the invention a straight-chain or branched alkyl radical having respectively 1 to 4 and 1 to 3 carbon atoms. A straight-chain alkyl radical having 1 to 3 carbon atoms is preferred. Examples which may be preferably mentioned are: methyl, ethyl, n-propyl, isopropyl, n-butyl, iso-butyl, sec-butyl, tert-butyl.

(C₁-C₄)-Alkoxy is in the context of the invention a straight-chain or branched alkoxy radical having 1 to 4 carbon atoms. Examples which may be preferably mentioned are: methoxy, ethoxy, n-propoxy, isopropoxy, n-butoxy, tert-butoxy.

(C₁-C₄)-Alkanediyl is in the context of the invention a straight-chain or branched divalent alkyl radical having 1 to 4 carbon atoms. A straight-chain alkanediyl radical having 2 to 4 carbon atoms is preferred. Examples which may be preferably mentioned are: methylene, 1,2-ethylene, ethane-1,1-diyl, 1,3-propylene, propane-1,1-diyl, propane-1,2-diyl, propane-2,2-diyl, 1,4-butylene, butane-1,2-diyl, butane-1,3-diyl, butane-2,3-diyl.

The side group of an α-amino acid in the meaning of R³ encompasses both the side groups of naturally occurring α-amino acids and the side groups of homologs and isomers of these α-amino acids. The α-amino acid may in this connection have both the L and the D configuration or else be a mixture of the L form and D form. Examples of side groups which may be mentioned are: hydrogen (glycine), methyl (alanine), propan-2-yl (valine), propan-1-yl(norvaline), 2-methylpropan-1-yl (leucine), 1-methylpropan-1-yl(isoleucine), butan-1-yl(norleucine), phenyl (2-phenylglycine), benzyl (phenylalanine), p-hydroxybenzyl (tyrosine), indol-3-ylmethyl (tryptophan), imidazol-4-ylmethyl (histidine), hydroxymethyl (serine), 2-hydroxyethyl (homoserine), 1-hydroxyethyl (threonine), mercaptomethyl (cysteine), methylthiomethyl (S-methylcysteine), 2-mercaptoethyl (homocysteine), 2-methylthioethyl (methionine), carbamoylmethyl (asparagine), 2-carbamoylethyl (glutamine), carboxymethyl (aspartic acid), 2-carboxyethyl (glutamic acid), 4-aminobutan-1-yl (lysine), 4-amino-3-hydroxybutan-1-yl(hydroxylysine), 3-aminopropan-1-yl (ornithine), 3-guanidinopropan-1-yl (arginine), 3-ureidopropan-1-yl (citrulline). Preferred α-amino acid side groups in the meaning of R³ are hydrogen (glycine), methyl (alanine), propan-2-yl (valine), propan-1-yl (norvaline), imidazol-4-ylmethyl (histidine), hydroxymethyl (serine), 1-hydroxyethyl (threonine), carbamoylmethyl (asparagine), 2-carbamoylethyl (glutamine), 4-aminobutan-1-yl (lysine), 3-aminopropan-1-yl(ornithine), 3-guanidinopropan-1-yl (arginine). The L configuration is preferred in each case.

If radicals in the compounds according to the invention are substituted, the radicals may, unless otherwise specified, be substituted one or more times. In the context of the present invention, all radicals which occur more than once have a mutually independent meaning. Substitution by one or two identical or different substituents is preferred. Substitution by one substituent is very particularly preferred.

Preference is given to compounds of the formula (I) in which

R¹ is hydrogen or (C₁-C₄)-alkyl,

R² is hydrogen,

and

L is a (C₂-C₄)-alkanediyl group or is a group of the formula

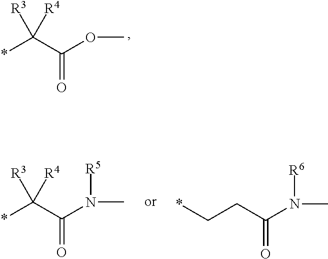

in which

* means the point of linkage to the N atom,

R³ is hydrogen, methyl, propan-2-yl, propan-1-yl, imidazol-4-ylmethyl, hydroxymethyl, 1-hydroxyethyl, carbamoylmethyl, 2-carbamoylethyl, 4-aminobutan-1-yl, 3-aminopropan-1-yl or 3-guanidinopropan-1-yl,

or

R³ is linked to R¹ and the two together form a (CH₂)₃ or (CH₂)₄ group,

R⁴ is hydrogen or methyl,

R⁵ is methyl,

and

R⁶ is hydrogen or methyl,

and the salts, solvates and solvates of the salts thereof.

Particularly important in this connection are compounds of the formula (I) in which

R¹ is hydrogen or (C₁-C₃)-alkyl.

Also particularly important are compounds of the formula (I) in which

L is a straight-chain (C₂-C₄)-alkanediyl group.

Particular preference is given to compounds of the formula (I) in which

R¹ is hydrogen, methyl or n-butyl,

R² is hydrogen,

and

L is a CH₂CH₂ group or is a group of the formula

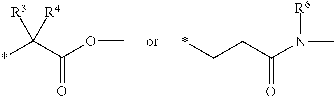

in which

* means the point of linkage to the N atom,

R³ is hydrogen, methyl, propan-2-yl, propan-1-yl, imidazol-4-ylmethyl, hydroxymethyl, 1-hydroxyethyl, carbamoylmethyl, 2-carbamoylethyl, 4-aminobutan-1-yl, 3-aminopropan-1-yl or 3-guanidinopropan-1-yl,

or

R³ is linked to R¹ and the two together form a (CH₂)₃ or (CH₂)₄ group,

R⁴ is hydrogen or methyl,

and

R⁶ is hydrogen or methyl,

and the salts, solvates and solvates of the salts thereof.

Particularly important in this connection are compounds of the formula (I) in which

R¹ is hydrogen or methyl.

Also particularly important are compounds of the formula (I) in which

L is a CH₂CH₂ group.

The invention further relates to a process for preparing the compounds according to the invention of the formula (I), characterized in that either

[A] the compound (A)

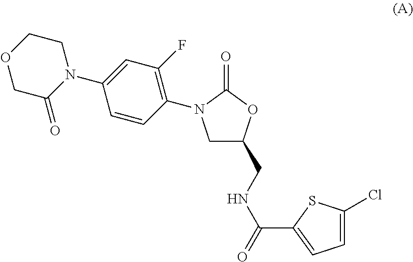

is initially converted in an inert solvent in the presence of a base with a compound of the formula (II)

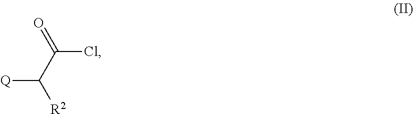

in which R² has the meaning indicated above,
and

Q is a leaving group such as, for example, chlorine, bromine or iodine, into a compound of the formula (III)

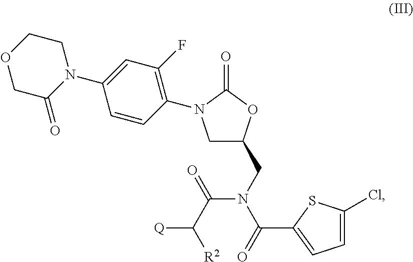

in which Q and R² have the meanings indicated above,
the latter is then reacted in an inert solvent with the cesium salt of an α-amino carboxylic acid or α-amino thiocarboxylic acid of the formula (IV)

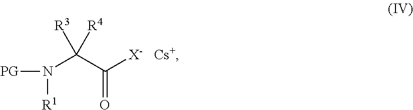

in which R¹, R³ and R⁴ each have the meanings indicated above,

PG is an amino protective group such as, for example, tert-butoxycarbonyl (Boc) or benzyloxycarbonyl (Z),

and

X is O or S,

to give a compound of the formula (V)

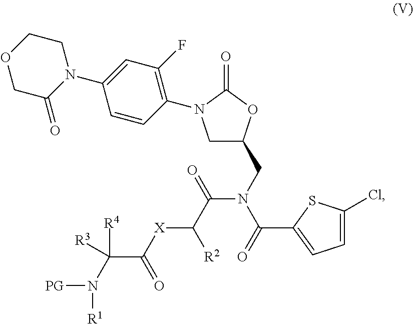

in which R¹, R², R³, R⁴, PG and X each have the meanings indicated above, and subsequently the protective group PG is removed by conventional methods to result in a compound of the formula (I-A)

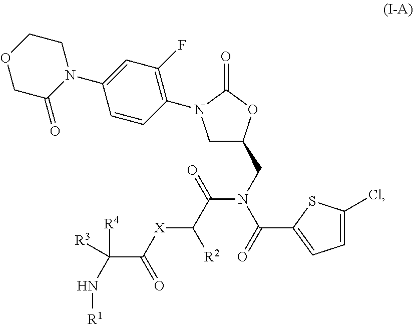

in which R¹, R², R³, R⁴ and X each have the meanings indicated above,
or

[B] compound (A) is reacted in an inert solvent in the presence of a base with a compound of the formula (VI)

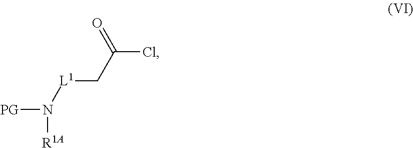

in which PG has the meaning indicated above, R^{1A} is (C₁-C₄)-alkyl which may be substituted by hydroxy or (C₁-C₄)-alkoxy, and

L¹ is a (C₁-C₄)-alkanediyl group in which one CH₂ group may be replaced by an O atom,

to give a compound of the formula (VII)

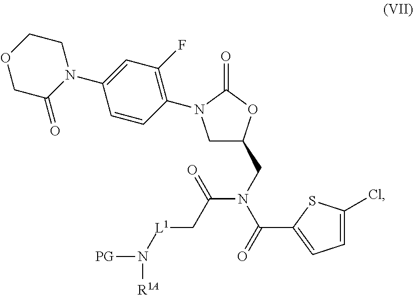

in which R^{1A}, L¹ and PG each have the meanings indicated above,
and subsequently the protective group PG is removed by conventional methods to result in a compound of the formula (I-B)

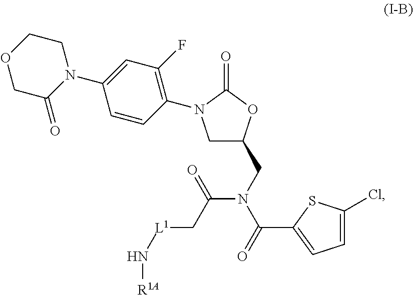

in which R^{1A} and L¹ have the meanings indicated above,
or
[C] the compound (B)

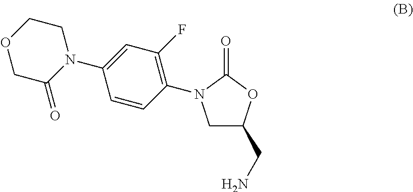

is initially converted by standard methods of peptide chemistry into a compound of the formula (VIII)

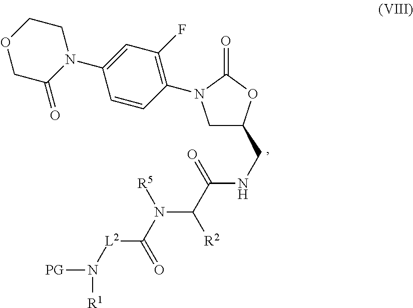

in which PG, R¹, R² and R⁵ each have the meanings indicated above,
and

L² is a (CH₂)₂ or CR³R⁴ group in which R³ and R⁴ each have the meanings indicated above,

the latter is then reacted in an inert solvent in the presence of a base with a compound of the formula (IX)

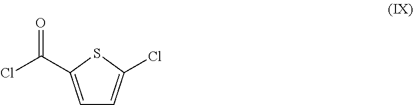

to give a compound of the formula (X)

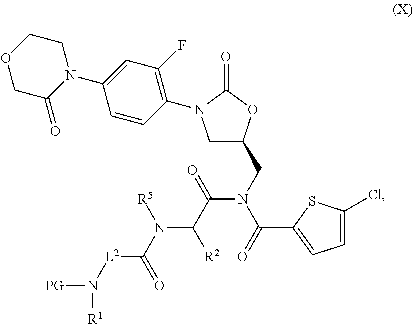

in which PG, L², R¹, R² and R⁵ each have the meanings indicated above, and subsequently the protective group PG is removed by conventional methods to result in a compound of the formula (I-C)

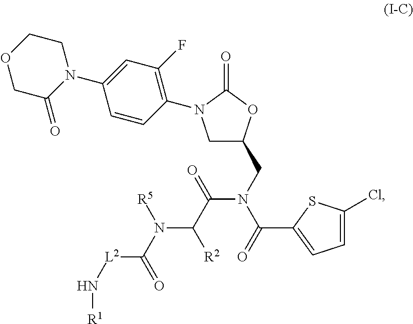

in which L², R¹, R² and R⁵ each have the meanings indicated above,
or

[D] compound (A) is reacted in an inert solvent in the presence of a base with a compound of the formula (XI)

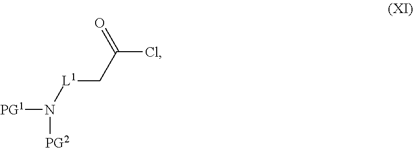

in which

L¹ is a (C₁-C₄)-alkanediyl group in which one CH₂ group may be replaced by an O atom,

and

PG¹ and PG² are independently of one another an amino protective group such as, for example, tert-butoxycarbonyl (Boc), benzyloxycarbonyl (Z) or p-methoxy-benzyl (PMB) and may be identical or different,

to give a compound of the formula (XII)

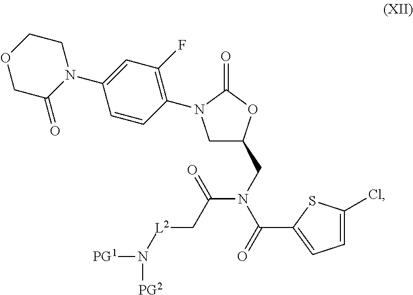

in which L¹, PG¹ and PG² each have the meanings indicated above, and subsequently the protective groups PG¹ and PG² are removed by conventional methods, simultaneously or sequentially, to result in a compound of the formula (I-D)

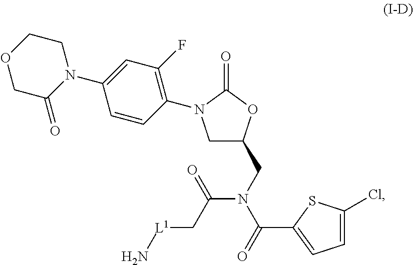

in which L¹ has the meaning indicated above,
and the compounds of the formula (I-A), (I-B), (I-C) and (I-D) resulting in each case are converted where appropriate with the appropriate (i) solvents and/or (ii) acids into the solvates, salts and/or solvates of the salts thereof.

The compounds of the formulae (I-A), (I-B), (I-C) and (I-D) may also result directly in the form of their salts in the preparation by the processes described above. These salts can be converted where appropriate by treatment with a base in an inert solvent, by chromatographic methods or by ion exchange resins, into the respective free bases.

Functional groups present where appropriate in the radicals R¹, R^{1A} and/or R³ may, if expedient or necessary, also be in temporarily protected form in the reaction sequences described above. The introduction and removal of such protective groups, as well as of the protective groups PG, PG¹ and PG², takes place in this connection by conventional methods known from peptide chemistry [see, for example, T. W. Greene and P. G. M. Wuts, Protective Groups in Organic Synthesis, Wiley, New York, 1999; M. Bodanszky and A. Bodanszky, The Practice of Peptide Synthesis, Springer-Verlag, Berlin, 1984].

Such protective groups which are present where appropriate in R¹, R^{1A} and/or R³ may in this connection be removed at the same time as the elimination of PG or in a separate reaction step before or after the elimination of PG.

The amino protective group PG, PG¹ or PG² preferably used in the above processes is tert-butoxycarbonyl (Boc), benzyloxycarbonyl (Z) or p-methoxybenzyl (PMB). Elimination of these protective groups is carried out by conventional methods, preferably by reacting with a strong acid such as hydrogen chloride, hydrogen bromide or trifluoroacetic acid in an inert solvent such as dioxane, dichloromethane or acetic acid; it is also possible where appropriate for the elimination to be carried out without an additional inert solvent.

The transformation (B)→(VIII) takes place by standard methods of peptide chemistry either by acylating the compound (B) with a suitably protected dipeptide derivative or by sequential coupling of the individual amino acid components, suitably protected where appropriate [cf., for example, M. Bodanszky, Principles of Peptide Synthesis, Springer-Verlag, Berlin, 1993; H.-D. Jakubke and H. Jeschkeit, Aminosäuren, Peptide, Proteine, Verlag Chemie, Weinheim, 1982].

The inert solvents preferably used in process steps (A)+(II)→(III), (A)+(VI)→(VII), (VIII)+(IX)→(X) and (A)+(XI)→(XII) are tetrahydrofuran, N,N-dimethylformamide or dimethyl sulfoxide; N,N-dimethylformamide is particularly preferred. A particularly suitable base in these reactions is sodium hydride. The reactions mentioned are generally carried out in a temperature range from 0° C. to +40° C. under atmospheric pressure.

Process step (III)+(IV)→(V) preferably takes place in N,N-dimethylformamide as solvent. The reaction is generally carried out in a temperature range from 0° C. to +50° C., preferably at +20° C. to +50° C., under atmospheric pressure. The reaction can also be carried out advantageously with ultrasound treatment.

The compounds of the formulae (II), (IV), (VI), (IX) and (XI) are commercially available, known from the literature or can be prepared by processes customary in the literature. Preparation of compound (A) is described in the Examples.

Preparation of the compounds according to the invention can be illustrated by the following synthesis schemes:

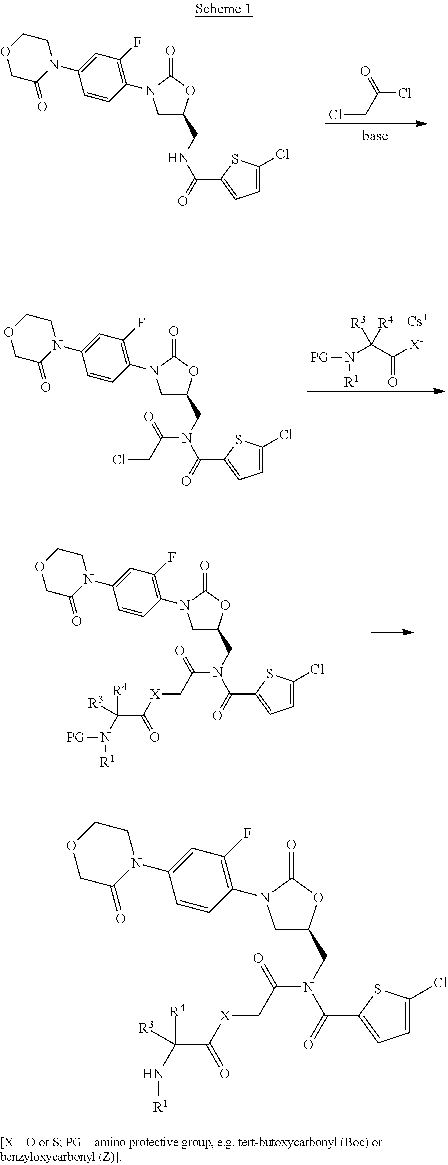

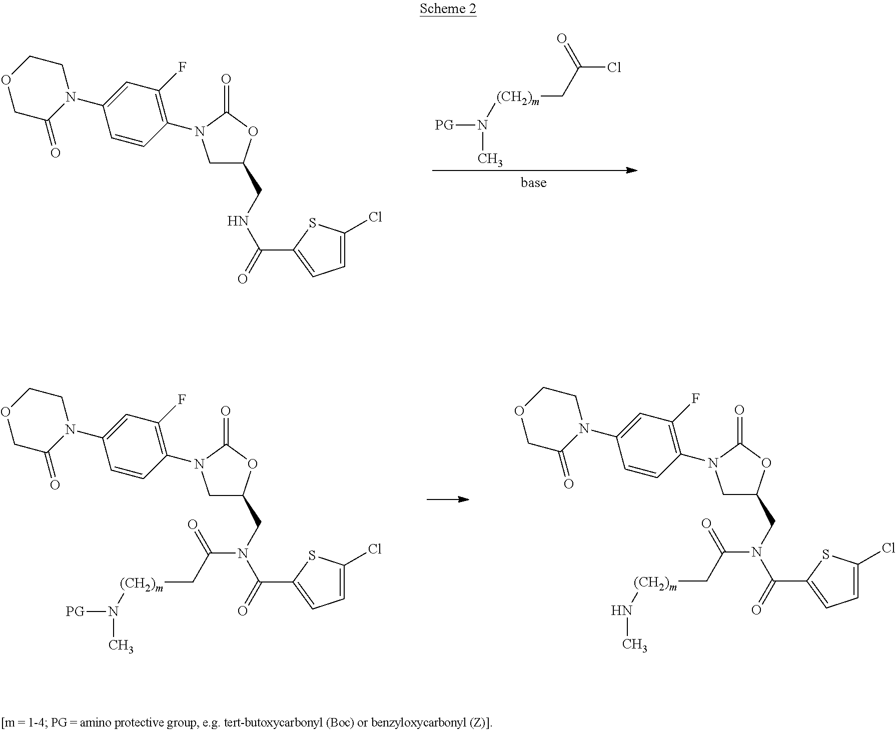

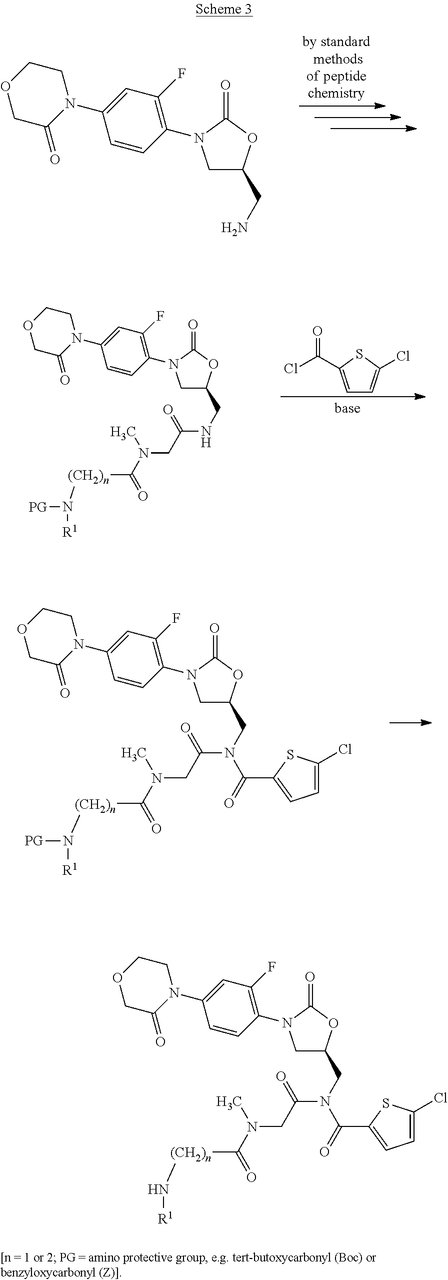

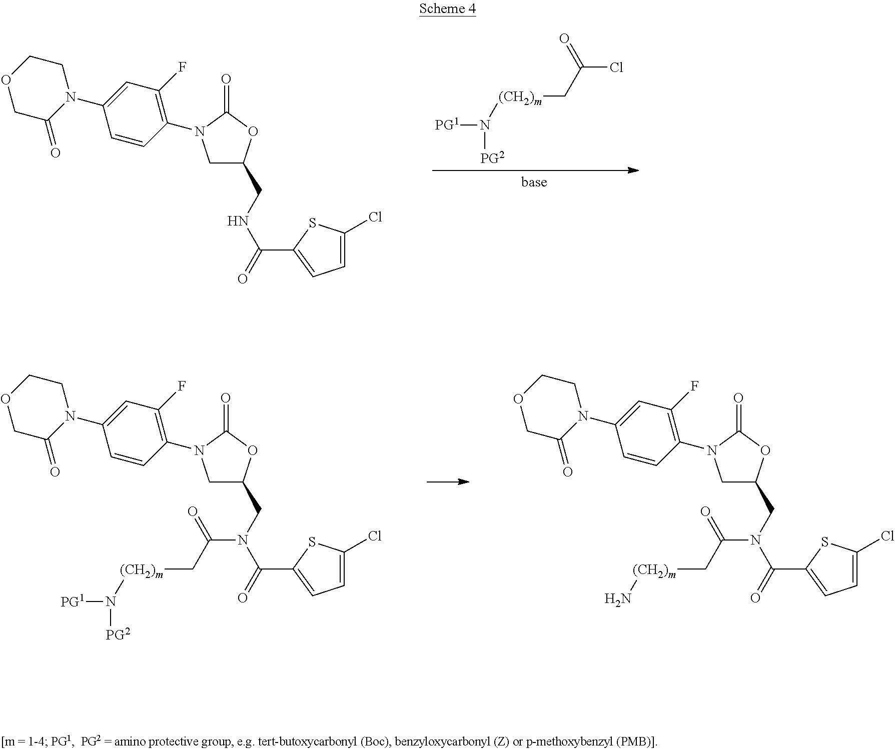

The compounds according to the invention and their salts represent useful prodrugs of the active ingredient compound (A). On the one hand, they show good stability at pH 4 and, on the other hand, they show efficient conversion into the active ingredient compound (A) at a physiological pH and in vivo. The compounds according to the invention moreover have good solubility in water and other physiologically tolerated media, making them suitable for therapeutic use especially on intravenous administration.

The present invention further relates to the use of the compounds according to the invention for the treatment and/or prophylaxis of disorders, preferably of thromboembolic disorders and/or thromboembolic complications.

The “thromboembolic disorders” include in the context of the present invention in particular disorders such as myocardial infarction with ST segment elevation (STEMI) and without ST segment elevation (non-STEMI), stable angina pectoris, unstable angina pectoris, reocclusions and restenoses following coronary interventions such as angioplasty or aortocoronary bypass, peripheral arterial occlusive diseases, pulmonary embolisms, deep venous thromboses and renal vein thromboses, transient ischemic attacks, and thrombotic and thromboembolic stroke.

The substances are therefore also suitable for the prevention and treatment of cardiogenic thromboembolisms, such as, for example, cerebral ischemias, stroke and systemic thromoboembolism and ischemias, in patients with acute, intermittent or persistent cardiac arrhythmias such as, for example, atrial fibrillation, and those undergoing cardioversion, also in patients with heart valve diseases or with artificial heart valves. The compounds according to the invention are additionally suitable for the treatment of disseminated intravascular coagulation (DIC).

Thromboembolic complications also occur in association with microangiopathic hemolytic anemia, extracorporeal circulations, such as hemodialysis, and heart valve prostheses.

The compounds according to the invention are additionally suitable also for the prophylaxis and/or treatment of atherosclerotic vascular disorders and inflammatory disorders such as rheumatic disorders of the musculoskeletal system, furthermore likewise for the prophylaxis and/or treatment of Alzheimer's disease. The compounds according to the invention can additionally be employed for inhibiting tumor growth and metastasis formation, for microangiopathies, age-related macular degeneration, diabetic retinopathy, diabetic nephropathy and other microvascular disorders, and for the prevention and treatment of thromoembolic complications such as, for example, venous thromboembolisms in tumor patients, especially those undergoing major surgical procedures or chemotherapy or radiotheraphy.

The present invention further relates to the use of the compounds according to the invention for the treatment and/or prophylaxis of disorders, especially of the aforementioned disorders.

The present invention further relates to the use of the compounds according to the invention for the manufacture of a medicament for the treatment and/or prophylaxis of disorders, especially of the aforementioned disorders.

The present invention further relates to a method for the treatment and/or prophylaxis of disorders, especially of the aforementioned disorders, using the compounds according to the invention.

The present invention further relates to medicaments comprising a compound according to the invention and one or more further active ingredients, especially for the treatment and/or prophylaxis of the aforementioned disorders. Examples of suitable combination active ingredients which may preferably be mentioned are:
lipid-lowering agents, especially HMG-CoA (3-hydroxy-3-methylglutarylcoenzyme A) reductase inhibitors;
coronary therapeutics/vasodilators, especially ACE (angiotensin converting enzyme) inhibitors, All (angiotensin II) receptor antagonists; β-adrenoceptorantagonists; alpha-1 adrenoceptor antagonists; diuretics; calcium channel blockers; substances which bring about an increase in cyclic guanosine monophosphate (cGMP), such as, for example, stimulators of soluble guanylate cyclase;
plasminogen activators (thrombolytics/fibrinolytics) and compounds which increase thrombolysis/fibrinolysis, such as inhibitors of plasminogen activator inhibitor (PAI inhibitors) or inhibitors of the thrombin-activated fibrinolysis inhibitor (TAFI inhibitors);
substances having anticoagulant activity (anticoagulants);
platelet aggregation-inhibiting substances (platelet aggregation inhibitors);
fibrinogen receptor antagonists (glycoprotein IIb/IIIa antagonists);
and antiarrhythmics.

The present invention further relates to medicaments which comprise at least one compound according to the invention, normally together with one or more inert, non-toxic, pharmaceutically suitable excipients, and to the use thereof for the aforementioned purposes.

The compounds according to the invention can act systemically and/or locally. For this purpose, they can be administered in a suitable way such as, for example, by the oral, parenteral, pulmonary or nasal route. The compounds according to the invention can be administered in administration forms suitable for these administration routes.

Suitable for oral administration are administration forms which function according to the prior art and deliver the compounds according to the invention rapidly and/or in modified fashion, and which contain the compounds according to the invention in crystalline and/or amorphized and/or dissolved form, such as, for example, tablets (uncoated or coated tablets, for example having enteric coatings or coatings which are insoluble or dissolve with a delay and control the release of the compound according to the invention), tablets which disintegrate rapidly in the mouth, or films/wafers, films/lyophilizates, capsules (for example hard or soft gelatin capsules), sugar-coated tablets, granules, pellets, powders, emulsions, suspensions, aerosols or solutions.

Parenteral administration can take place with avoidance of an absorption step (e.g. intravenous, intraarterial, intracardiac, intraspinal or intralumbar) or with inclusion of an absorption (e.g. intramuscular, subcutaneous, intracutaneous, percutaneous or intraperitoneal). Administration forms suitable for parenteral administration are, inter alia, preparations for injection and infusion in the form of solutions, suspensions, emulsions, lyophilizates or sterile powders.

Suitable for the other administration routes are, for example, pharmaceutical forms for inhalation, such as power inhalers or nebulizers, or pharmaceutical forms which can be administered nasally, such as drops, solutions or sprays.

Parenteral administration is preferred, especially intravenous administration.

The compounds according to the invention can be converted into the stated administration forms. This can take place in a manner known per se by mixing with inert, non-toxic, pharmaceutically suitable excipients. These excipients include, inter alia, carriers (for example microcrystalline cellulose, lactose, mannitol), solvents (e.g. liquid polyethylene glycols), emulsifiers and dispersants or wetting agents (for example sodium dodecyl sulfate, polyoxysorbitan oleate), binders (for example polyvinylpyrrolidone), synthetic and natural polymers (for example albumin), stabilizers (e.g. antioxidants such as, for example, ascorbic acid), colorants (e.g. inorganic pigments such as, for example, iron oxides) and masking flavors and/or odors.

It has generally proved advantageous to administer on parenteral administration amounts of about 0.001 to 1 mg/kg, preferably about 0.01 to 0.5 mg/kg, of body weight to achieve effective results, and on oral administration the dosage is about 0.01 to 100 mg/kg, preferably about 0.01 to 20 mg/kg, and very particularly preferably 0.1 to 10 mg/kg, of body weight.

It may nevertheless be necessary where appropriate to deviate from the stated amounts, in particular as a function of the body weight, route of administration, individual response to the active ingredient, nature of the preparation and time or interval over which administration takes place. Thus, it may be sufficient in some cases to make do with less than the aforementioned minimum amount, whereas in other cases the stated upper limit must be exceeded. It may in the event of administration of larger amounts be advisable to divide these into a plurality of individual doses over the day.

The following exemplary embodiments illustrate the invention. The invention is not restricted to the examples.

The percentage data in the following tests and examples are, unless indicated otherwise, percentages by weight; parts are parts by weight. Solvent ratios, dilution ratios and concentration data for the liquid/liquid solutions are in each case based on volume.

### A. EXAMPLES

### Abbreviations and Acronyms

abs. absolute

Boc tert-butoxycarbonyl

DMF N,N-dimethylformamide

DMSO dimethyl sulfoxide

h hour(s)

HPLC high pressure, high performance liquid chromatography

LC-MS coupled liquid chromatography-mass spectrometry

min minute(s)

MS mass spectrometry

NMR nuclear magnetic resonance spectrometry

p para

Pd/C palladium on activated carbon

PMB p-methoxybenzyl

quant. quantitative (for yield)

R_{f} retention index (for TLC)

RT room temperature

Rₜ retention time (for HPLC)

TLC thin-layer chromatography

UV ultraviolet spectrometry

v/v volume to volume ratio (of a solution)

Z benzyloxycarbonyl

LC-MS and HPLC methods:

Method 1: Instrument: HP 1100 with DAD detection; column: Kromasil 100 RP-18, 60 mm×2.1 mm, 3.5 μm; mobile phase A: 5 ml of perchloric acid (70% strength)/l of water, mobile phase B: acetonitrile; gradient: 0 min 2% B→0.5 min 2% B→4.5 min 90% B→6.5 min 90% B→6.7 min 2% B→7.5 min 2% B; flow rate: 0.75 ml/min; column temperature: 30° C.; UV detection: 210 nm.

Method 2: Instrument: HP 1100 with DAD detection; column: Kromasil 100 RP-18, 60 mm×2.1 mm, 3.5 μm; mobile phase A: 5 ml of perchloric acid (70% strength)/l of water, mobile phase B: acetonitrile; gradient: 0 min 2% B→0.5 min 2% B→4.5 min 90% B→9 min 0% B→9.2 min 2% B→10 min 2% B; flow rate: 0.75 ml/min; column temperature: 30° C.; UV detection: 210 nm.

Method 3 (LC-MS): MS instrument type: Micromass ZQ; HPLC instrument type: HP 1100 Series; UV DAD; column: Phenomenex Gemini 3μ 30 mm×3.00 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

Method 4 (LC-MS): Instrument: Micromass GCT, GC6890; column: Restek RTX-35MS, 30 m×250 μm×0.25 μm; constant helium flow rate: 0.88 ml/min; oven: 60° C.; inlet: 250° C.; gradient: 60° C. (maintained for 0.30 min), 50° C./min→120° C., 16° C./min→250° C., 30° C./min→300° C. (maintained for 1.7 min).

Method 5 (preparative HPLC): column: GROM-SIL 120 ODS-4 HE, 10 μM, 250 mm×30 mm; flow rate: 50 ml/min; mobile phase and gradient program: acetonitrile/0.1% aqueous formic acid 10:90 (0-3 min), acetonitrile/0.1% aqueous formic acid 10:90→95:5 (3-27 min), acetonitrile/0.1% aqueous formic acid 95:5 (27-34 min), acetonitrile/0.1% aqueous formic acid 10:90 (34-38 min); temperature: 22° C.; UV detection: 254 nm.

Method 6 (LC-MS): Instrument: Micromass LCT with HPLC Agilent Series 1100; column: Waters Symmetry C18, 3.5 μm, 50 mm×2.1 mm; mobile phase A: 1 l of water+1 ml of 98-100%-strength formic acid, mobile phase B: 1 l of acetonitrile+1 ml of 98-100% strength formic acid; gradient: 0 min 100% A→1 min 100% A→6 min 10% A→8 min 0% A→10 min 0% A→10.1 min 100% A→>12 min 100% A; flow rate: 0-10 min 0.5 ml/min→10.1 min 1 ml/min→>12 min 0.5 ml/min; temperature: 40° C.; UV detection DAD: 208-500 nm.

Method 7 (analytical HPLC): Instrument: WATERS 2695 with DAD996; column: XTerra 3.9×150 WAT 186000478; mobile phase A: 10 ml of 70% strength perchloric acid in 2.5 liters of water, mobile phase B: acetonitrile; gradient: 0.0 min 20% B→1 min 20% B→4 min 90% B→9 min 90% B; temperature: RT; flow rate: 1 ml/min.

Method 8 (LC-MS): Instrument: Micromass Quattro LCZ with HPLC Agilent Series 1100; column: Phenomenex Onyx Monolithic C18, 100 mm×3 mm; mobile phase A: 1 l of water+0.5 ml of 50% strength formic acid, mobile phase B: 1 l of acetonitrile+0.5 ml of 50% strength formic acid; gradient: 0.0 min 90% A→2 min 65% A→4.5 min 5% A→6 min 5% A; flow rate: 2 ml/min; oven: 40° C.; UV detection: 208-400 nm.

Method 9 (LC-MS): MS instrument type: Waters (Micromass) Quattro Micro; HPLC instrument type: Agilent 1100 Series; column: Thermo Hypersil GOLD 3μ 20 mm×4 mm; mobile phase A: 1 l of water+0.5 ml of 50% strength formic acid, mobile phase B: 1 l of acetonitrile+0.5 ml of 50% strength formic acid; gradient: 0.0 min 100% A→3.0 min 10% A→4.0 min 10% A→4.01 min 100% A (flow rate 2.5 ml)→5.00 min 100% A; oven: 50° C.; flow rate: 2 ml/min; UV detection: 210 nm.

Method 10 (LC-MS): MS instrument type: Micromass ZQ; HPLC instrument type: Waters Alliance 2795; column: Phenomenex Synergi 2.5μ MAX-RP 100A Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→0.1 min 90% A→3.0 min 5% A→4.0 min 5% A→4.01 min 90% A; flow rate: 2 ml/min; oven: 50° C.; UV detection: 210 nm.

Method 11 (analytical HPLC): Instrument: HP1090 Series II; column: Waters XTerra 018-5, 3.9 mm×150 mm WAT 186000478; mobile phase A: 10 ml of 70% strength perchloric acid in 2.5 l of water, mobile phase B: acetonitrile; gradient: 0.0 min 20% B→1 min 20% B→4 min 90% B→6 min 90% B→8 min 20% B. temperature: 40° C.; flow rate: 1 ml/min.

Method 12 (analytical HPLC): Instrument: HP 1090 Series II; column: Merck Chromolith Speed ROD RP-18e, 50 mm×4.6 mm; precolumn Chromolith Guard Cartridge Kit, RP-18e, 5-4.6 mm; mobile phase A: 5 ml of perchloric acid (70% strength)/ I of water, mobile phase B: acetonitrile; gradient: 0 min 20% B→0.5 min 20% B→3 min 90% B→3.5 min 90% B→3.51 min 20% B→4 min 20% B; flow rate: 5 ml/min; column temperature: 40° C.; UV detection: 210 nm.

Method 13 (preparative HPLC): Instrument: Gilson with UV detector, column: Kromasil C18, 5 μm/250 mm×20 mm (flow rate: 25 ml/min); mobile phase A: water (0.01% trifluoroacetic acid), mobile phase B: acetonitrile (0.01% trifluoroacetic acid); gradient: 0 min 5-20% B, 10 min-15 min 5-20% B, 45 min 90% B, 50 min 90% B; flow rate: 25 ml/min; UV detection: 210 nm.

Method 14 (preparative HPLC): Instrument: Gilson with UV detector, column: YMC ODS AQ C18, 10 μm/250 mm×30 mm (flow rate: 50 ml/min); mobile phase A: water (0.01% trifluoroacetic acid), mobile phase B: acetonitrile (0.01% trifluoroacetic acid); gradient: 0 min 5-20% B, 10 min-15 min 5-20% B, 45 min 90% B, 50 min 90% B; flow rate: 50 ml/min; wavelength: 210 nm.

### NMR Spectrometry:

NMR measurements were carried out at a proton frequency of 400.13 MHz. The samples were normally dissolved in DMSO-d₆; temperature: 302 K.

### Starting Compounds and Intermediates:

The starting material used was 5-chloro-N-({(5S)-3-[2-fluoro-4-(3-oxomorpholin-4-yl)phenyl]-2-oxo-1,3-oxazolidin-5-yl}methyl)thiophene-2-carboxamide [compound (A)].

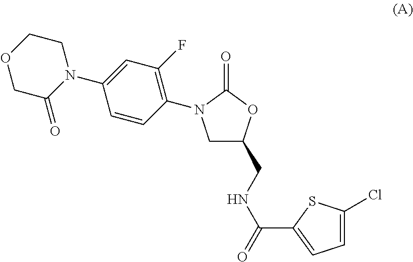

### Example 1A

### 5-Chlorothiophene-2-carbonyl chloride

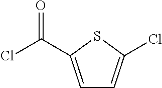

137 ml (1.57 mol) of oxalyl chloride were added to a suspension of 51.2 g (0.315 mmol) of 5-chlorothiophene-2-carboxylic acid in 307 ml of dichloromethane. After addition of 2 drops of DMF the mixture was stirred at room temperature for 15 hours. The solvent and excess oxalyl chloride were then removed on a rotary evaporator. The residue was distilled under reduced pressure. The product boiled at 74-78° C. and a pressure of 4-5 mbar. This gave 50.5 g (87% of theory) of an oil which solidified on storage in the fridge.

¹H-NMR (400 MHz, CDCl₃, 6/ppm): 7.79 (d, 1H), 7.03 (d, 1H).

GC/MS (Method 4): Rₜ=5.18 min.

MS (EI+, m/z): 180/182/184 (2 ³⁵Cl/³⁷Cl) M⁺.

### Example 2A

### ((S)-2,3-Dihydroxypropyl)-5-chlorothiophene-2-carboxamide

(from: C. R. Thomas, Bayer HealthCare AG, DE-10300111-A1 (2004).)

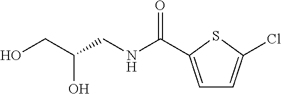

At 13-15° C., 461 g (4.35 mol) of sodium bicarbonate and 350 g (3.85 mol) of (2S)-3-aminopropane-1,2-diol hydrochloride were initially charged in 2.1 l of water, and 950 ml of 2-methyltetrahydrofuran were added. With cooling at 15-18° C., 535 g (2.95 mol) of 5-chlorothiophene-2-carbonyl chloride (compound from Example 1A) in 180 ml of toluene were added dropwise to this mixture over a period of two hours. For work-up, the phases were separated and a total of 1.5 l of toluene was added in a plurality of steps to the organic phase. The precipitated product was filtered off with suction, washed with ethyl acetate and dried. This gave 593.8 g (92% of theory) of product.

### Example 3A

### ((S)-3-Bromo-2-hydroxypropyl)-5-chlorothiophene-2-carboxamide

(from: C. R. Thomas, Bayer HealthCare AG, DE-10300111-A1 (2004).)

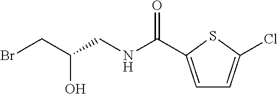

Over a period of 30 minutes, 301.7 ml of a 33% strength solution of hydrogen bromide in acetic acid were, at 21-26° C., added to a suspension of 100 g (0.423 mol) of the compound from Example 2A in 250 ml of glacial acetic acid. 40 ml of acetic anhydride were then added, and the reaction mixture was stirred at 60-65° C. for three hours. At 20-25° C., 960 ml of methanol were then added over a period of 30 minutes. The reaction mixture was stirred under reflux for 2.5 hours and then at 20-25° C. overnight. For work-up, the solvents were distilled off under reduced pressure at about 95 mbar. 50 ml of n-butanol and 350 ml of water were added to the suspension that remained. The precipitated product was filtered off with suction, washed with water and dried. This gave 89.8 g (71% of theory) of product.

### Example 4A

### 5-Chloro-N-[(2S)-oxiran-2-ylmethyl]thiophene-2-carboxamide

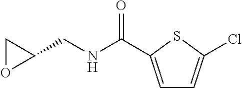

155 g (1.12 mol) of powdered potassium carbonate were added to a solution of 50 g (0.167 mol) of the compound from Example 3A in 500 ml of anhydrous THF, and the mixture was stirred at room temperature for 3 days. The inorganic salts were then filtered off with suction over a layer of kieselguhr and washed twice with in each case 100 ml of THF, and the filtrate was concentrated on a rotary evaporator at room temperature. This gave 36 g (81% of theory) of product.

¹H-NMR (400 MHz, DMSO-d₆, δ/ppm): 8.81 (t, 1H), 7.68 (d, 1H), 7.19 (d, 1H), 3.55-3.48 (m, 1H), 3.29-3.22 (m, 1H), 3.10-3.06 (m, 1H), 2.75-2.72 (m, 1H), 2.57-2.54 (m, 1H).

HPLC (Method 1): Rₜ=3.52 min.

MS (DCl, NH₃, m/z): (³⁵Cl/³⁷Cl) 218/220 (M+H)⁺, 235/237 (M+NH₄)⁺.

### Example 5A

### N,N-Dibenzyl-2-fluoro-4-iodoaniline

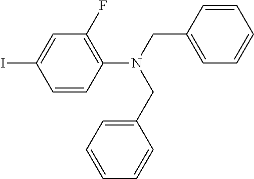

In a mixture of 100 ml of water and 200 ml of dichloromethane, 24.37 g (0.103 mol) of 2-fluoro-4-iodoaniline, 31.8 ml (0.267 mol) of benzyl bromide, 23.98 g (0.226 mol) of sodium carbonate and 1.9 g (5.14 mmol) of tetra-n-butylammonium iodide were heated at reflux for six days. After cooling to room temperature, the phases were separated from one another. The organic phase was washed with water and saturated sodium chloride solution and dried over anhydrous sodium sulfate. After filtration, the solvent was removed on a rotary evaporator. The residue obtained was purified by filtration with suction through silica gel using the mobile phase cyclohexane. This gave 35 g (82% of theory) of the title compound.

¹H-NMR (400 MHz, DMSO-d₆, δ/ppm): 7.48 (1H, dd), 7.32-7.21 (m, 11H), 6.69 (dd, 1H), 4.33 (s, 4H).

HPLC (Method 1): Rₜ=5.87 min.

MS (DCl, NH₃, m/z): 418 (M+H)⁺.

### Example 6A

### 4-[4-(Dibenzylamino)-3-fluorophenyl]morpholin-3-one

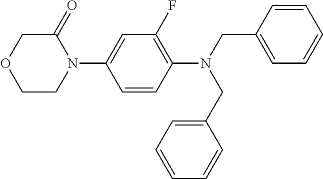

1.5 g (3.59 mmol) of the compound from Example 5A were dissolved in 20 ml of anhydrous dioxane, and 0.45 g (4.49 mmol) of morpholinone, 137 mg (0.719 mmol) of copper(I) iodide, 1.53 g (7.19 mmol) of potassium phosphate and 153 μl (1.44 mmol) of N,N′-dimethylethylenediamine were added in succession. The reflux apparatus was inertized by repeated application of a slightly reduced pressure and venting with argon. The reaction mixture was heated at reflux for 15 hours. After this period of time, the mixture was allowed to cool to room temperature. Water was added, and the mixture was extracted with ethyl acetate.

The organic extract was washed successively with water and saturated sodium chloride solution. The extract was dried over anhydrous magnesium sulfate and then filtered, and the filtrate was freed from the solvent under reduced pressure. The residue was purified by filtration with suction through silica gel using the mobile phase cyclohexane/ethyl acetate 1:1. This gave 1.38 g (98% of theory) of the title compound.

¹H-NMR (400 MHz, DMSO-d₆, δ/ppm): 7.32-7.28 (m, 9H), 7.26-7.20 (m, 2H), 7.00-6.92 (m, 2H), 4.33 (s, 4H), 4.15 (s, 2H), 3.91 (dd, 2H), 3.55 (dd, 2H).

HPLC (Method 1): Rₜ=4.78 min.

MS (DCl, NH₃, m/z): 391 (M+H)⁺.

### Example 7A

### 4-(4-Amino-3-fluorophenyl)morpholin-3-one

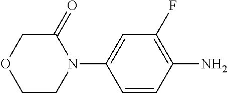

### Method 1:

700 mg (1.79 mmol) of the compound from Example 6A were dissolved in 70 ml of ethanol, and 95 mg of palladium on activated carbon (10%) were added. The mixture was hydrogenated at room temperature and a hydrogen pressure of 1 bar for one hour. The catalyst was then filtered off through a little kieselguhr and the filtrate was concentrated on a rotary evaporator. This gave 378 mg (95% of theory) of the title compound.

¹H-NMR (400 MHz, DMSO-d₆, δ/ppm): 7.04 (dd, 1H), 6.87 (dd, 1H), 6.73 (dd, 1H), 5.17 (s, broad, 2H), 4.12 (s, 2H), 3.91 (dd, 2H), 3.62 (dd, 2H).

HPLC (Method 1): Rₜ=0.93 min.

MS (DCl, NH₃, m/z): 211 (M+H)⁺, 228 (M+NH₄)⁺.

### Method 2:

Under argon, a suspension of 29.6 g (125 mmol) of 2-fluoro-4-iodoaniline, 15.8 g (156 mmol, 1.25 eq.) of morpholin-3-one [J.-M. Lehn, F. Montavon, Helv. Chim. Acta 1976, 59, 1566-1583], 9.5 g (50 mmol, 0.4 eq.) of copper(I) iodide, 53.1 g (250 mmol, 2 eq.) of potassium phosphate and 8.0 ml (75 mmol, 0.6 eq.) of N,N′-dimethylethylenediamine in 300 ml of dioxane was stirred under reflux overnight. After cooling to RT, the reaction mixture was filtered through a layer of kieselguhr and the residue was washed with dioxane. The combined filtrates were concentrated under reduced pressure. The crude product was purified by flash chromatography (silica gel 60, dichloromethane/methanol 100:1→100:3). This gave 24 g (74% of theory) of the title compound.

LC-MS (Method 3): Rₜ=0.87 min;

MS (ESIpos): m/z=211 [M+H]⁺;

¹H-NMR (500 MHz, DMSO-d₆): δ=7.05 (dd, 1H), 6.87 (dd, 1H), 6.74 (dd, 1H), 5.14 (s, 2H), 4.11 (s, 2H), 3.92 (dd, 2H), 3.63 (dd, 2H).

### Example 8A

### 5-Chloro-N-[(2R)-3-{[2-fluoro-4-(3-oxomorpholin-4-yl)phenyl]amino}-2-hydroxypropyl]thiophene-2-carboxamide

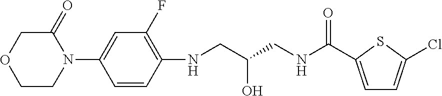

600 mg (2.69 mmol) of magnesium perchlorate were added to a solution of 376 mg (1.79 mmol) of the product from Example 7A and 429 mg (1.97 mmol) of the compound from Example 4A in 10 ml of acetonitrile, and the mixture was stirred at room temperature for 15 hours. Water was added, and the mixture was extracted with ethyl acetate. The organic extract was washed successively with water and saturated sodium chloride solution and dried over anhydrous magnesium sulfate. After filtration, the solvent was removed on a rotary evaporator. The residue was purified by preparative HPLC (Method 5). This gave 503 mg (64% of theory) of the title compound.

¹H-NMR (400 MHz, DMSO-d₆, δ/ppm): 8.61 (t, 1H), 7.68 (d, 1H), 7.18 (d, 1H), 7.11 (dd, 1H), 6.97 (dd, 1H), 6.73 (dd, 1H), 5.33 (t, 1H), 5.14 (d, 1H), 4.13 (s, 2H), 3.92 (dd, 2H), 3.87-3.79 (m, 1H), 3.63 (dd, 2H), 3.39-3.22 (m, 2H, partly superposed by the water signal), 3.21-3.15 (m, 1H), 3.08-3.02 (m, 1H).

HPLC (Method 1): Rₜ=3.75 min.

MS (DCl, NH₃, m/z): 428/430 (³⁵Cl/³⁷Cl) (M+H)⁺, 445/447 (M+NH₄)⁺.

### Example 9A

### Compound A

### 5-Chloro-N-({(5S)-3-[2-fluoro-4-(3-oxomorpholin-4-yl)phenyl]-2-oxo-1,3-oxazolidin-5-yl}methyl)thiophene-2-carboxamide

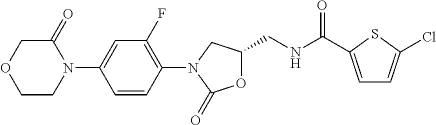

2.7 mg (0.022 mmol) of 4-dimethylaminopyridine were added to a solution of 478 mg (1.12 mmol) of the product from Example 8A and 363 mg (2.24 mmol) of carbonyldiimidazole in 10 ml of butyronitrile, and the mixture was heated at 70° C. After three days, the solvent was removed on a rotary evaporator. The product was isolated from the residue by preparative HPLC (Method 5). This gave 344 mg (68% of theory) of the title compound.

¹H-NMR (400 MHz, DMSO-d₆, δ/ppm): 8.98 (t, 1H), 7.70 (d, 1H), 7.52 (dd, 1H), 7.48 (dd, 1H), 7.31 (dd, 1H), 7.21 (d, 1H), 4.91-4.84 (m, 1H), 4.21 (s, 2H), 4.12 (t, 1H), 3.98 (dd, 2H), 3.80 (dd, 1H), 3.76 (dd, 2H), 3.68-3.57 (m, 2H).

HPLC (Method 1): Rₜ=3.82 min.

MS (DCl, NH₃, m/z): 471/473 (³⁵Cl/³⁷Cl) (M+NH₄)⁺.

### Example 10A

### 5-Chloro-N-(chloroacetyl)-N-({(5S)-2-oxo-3-[2-fluoro-4-(3-oxomorpholin-4-yl)phenyl]-1,3-oxa-zolidin-5-yl}methyl)thiophene-2-carboxamide

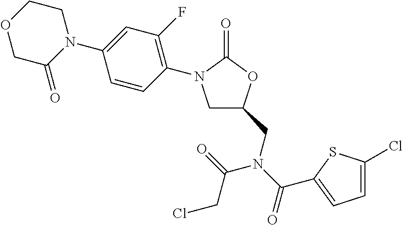

The compound can be prepared analogously to steps a) in Example 13, 19 or 22 by reacting the compound (A) with chloroacetyl chloride.

### Example 11A

### Benzyl (4-chloro-4-oxobutyl)methylcarbamate

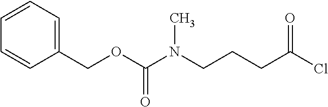

Initially, 4-[[(benzyloxy)carbonyl](methyl)amino]butyric acid was prepared by introducing the benzyloxycarbonyl protective group into the corresponding ω-N-methylaminoalkylcarboxylic acid, which can be obtained according to P. Quitt et al. [Helv. Chim. Acta 46, 327 (1963)]. Alternatively, 4-[[(benzyloxy)carbonyl](methyl)amino]butyric acid can also be prepared according to a literature procedure [Y. Aramaki et al., Chem. Pharm. Bull. 52, 258 (2004)] from commercially available 4-{[(benzyloxy)carbonyl]amino}butyric acid. 1.74 g (6.92 mmol) of 4-[[(benzyloxy)carbonyl](methyl)amino]butyric acid were dissolved in 35 ml of dichloromethane, and 3.5 ml (48 mmol) of thionyl chloride were added. The mixture was heated under reflux for 1 h. The mixture was then concentrated under reduced pressure, more dichloromethane was added to the residue and the mixture was concentrated again. What remained was a viscous oil which was dried under high vacuum. This gave 1.8 g (96% of theory) of the target compound which was further reacted without further purification and characterization.

### Example 12A

### Benzyl (5-chloro-5-oxopentyl)methylcarbamate

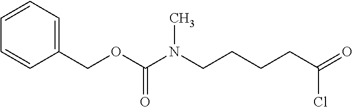

Initially, 5-[[(benzyloxy)carbonyl](methyl)amino]valeric acid was prepared according to known methods. Here, the benzyloxycarbonyl protective group was introduced into ω-N-methylaminovaleric acid which had been prepared beforehand by reaction of ω-bromovaleric acid with methylamine.

1.97 g (7.43 mmol) of 5-[[(benzyloxy)carbonyl](methyl)amino]valeric acid were dissolved in 30 ml of dichloromethane, and 4.9 ml (67.3 mmol) of thionyl chloride were added. The mixture was heated under reflux for 1 h. The mixture was then concentrated under reduced pressure, more dichloromethane was added to the residue and the mixture was concentrated again. What remained was a viscous oil which was dried under high vacuum. This gave 2 g (95% of theory) of the target compound which was further reacted without further purification and characterization.

### Example 13A

### Benzyl (3-chloro-3-oxopropyl)methylcarbamate

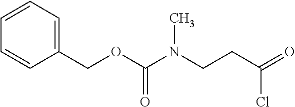

Initially, 3-[[(benzyloxy)carbonyl](methyl)amino]propionic acid was prepared by introducing the benzyloxycarbonyl protective group into the corresponding ω-N-methylaminoalkylcarboxylic acid which can be obtained according to P. Quitt et al. [Helv. Chim. Acta 46, 327 (1963)]. Alternatively, 3-[[(benzyloxy)carbonyl](methyl)amino]propionic acid can be prepared according to a literature procedure [Y. Aramaki et al., Chem. Pharm. Bull. 52, 258 (2004)] from commercially available 3-{[(benzyloxy)carbonyl]amino}propionic acid.

850 mg (3.58 mmol) of 3-[[(benzyloxy)carbonyl](methyl)amino]propionic acid were dissolved in 15 ml of dichloromethane and 1.5 ml of oxalyl chloride were added. The mixture was heated under reflux for 3 h. The mixture was then concentrated under reduced pressure, more dichloromethane was added to the residue and the mixture was concentrated again. What remained was a viscous oil which was dried under high vacuum. This gave 915 mg (quant.) of the target compound which was further reacted without further purification and characterization.

### Example 14A

### Benzyl (6-chloro-6-oxohexyl)methylcarbamate

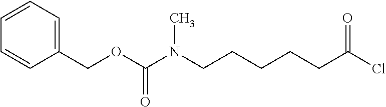

Initially, 6-[[(benzyloxy)carbonyl](methyl)amino]caproic acid was prepared by introducing the benzyloxycarbonyl protective group into the corresponding ω-N-methylaminoalkylcarboxylic acid which can be obtained according to P. Quitt et al. [Helv. Chim. Acta 46, 327 (1963)]. Alternatively, 6-[[(benzyloxy)carbonyl](methyl)amino]caproic acid can be prepared according to a literature procedure [Y. Aramaki et al., Chem. Pharm. Bull. 52, 258 (2004)] from commercially available 6-{[(benzyloxy)carbonyl]amino}caproic acid.

3850 mg (13.8 mmol) of 6-[[(benzyloxy)carbonyl](methyl)amino]caproic acid were dissolved in 60 ml of dichloromethane and 4 ml of oxalyl chloride were added. The mixture was heated under reflux for 3 h. The mixture was then concentrated under reduced pressure, more dichloromethane was added to the residue and the mixture was concentrated again. What remained was a viscous oil which was dried under high vacuum. This gave 4.1 g (quant.) of the target compound which was reacted without further purification and characterization.

### Example 15A

### Benzyl (5-chloro-5-oxopentyl)(4-methoxybenzyl)carbamate

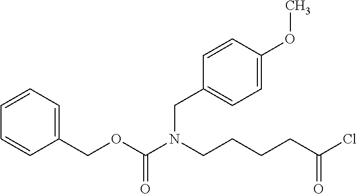

### Step a):

10 g (85.4 mmol) of 5-aminovaleric acid, 17.4 g (128 mmol) of p-anisaldehyde and 10.3 g (85.4 mmol) of magnesium sulfate were taken up in 330 ml of ethanol and heated under reflux for 1 h. The mixture was then filtered off, the filter residue was washed with ethanol and a total of 1.94 g (51.2 mmol) of sodium borohydride was then added a little at a time over a period of 15 min to the filtrate. Initially, 10 ml of water were added, and then 128 ml of a 2 M aqueous sodium hydroxide solution. After 5 min, the mixture was diluted with 300 ml of water and then extracted three times with in each case 200 ml of ethyl acetate. The aqueous phase was adjusted to pH 2 using 4 M hydrochloric acid and concentrated under reduced pressure. The residue was purified by flash chromatography on silica gel using the mobile phase acetonitrile/water/acetic acid 5:1:0.1. The product fractions were concentrated and triturated with ethyl acetate and diethyl ether. The residue was then filtered off with suction and dried under high vacuum. This gave 9.1 g (45% of theory) of the p-methoxybenzyl-protected 5-aminovaleric acid.

### Step b):

The 5-aminovaleric acid derivative obtained in this manner was taken up in dioxane/water (1:1) and adjusted to pH 10 using aqueous sodium hydroxide solution, and 12.97 g (76 mmol) of benzyl chlorocarbonate were then added dropwise. After 15 min of stirring at RT, the dioxane was removed under reduced pressure and the solution that remained was adjusted to pH 2 using 2 M hydrochloric acid. The mixture was extracted with ethyl acetate and the organic phase was then washed twice with water. The organic phase was then concentrated and the residue was dried under high vacuum. This was followed by purification by flash chromatography on silica gel using the mobile phase acetonitrile. The product fractions were concentrated and the residue was dried under high vacuum. This gave 5.6 g (38% of theory) of the Z-protected amino acid.

LC-MS (Method 6): Rₜ=2.47 min; m/z=372 (M+H)⁺.

### Step c):

5.6 g (15 mmol) of the 5-{[(benzyloxy)carbonyl](4-methoxybenzyl)amino}valeric acid obtained in this manner were dissolved in 60 ml of dichloromethane and 2.2 ml of thionyl chloride were added. The mixture was heated under reflux for 30 min. The mixture was then concentrated under reduced pressure, more dichloromethane was added to the residue and the mixture was concentrated again. What remained was a viscous oil which was dried under high vacuum. This gave 5.7 g (98% of theory) of the target compound which was further reacted without further purification and characterization.

### Example 16A

### Benzyl (6-chloro-6-oxohexyl)(4-methoxybenzyl)carbamate

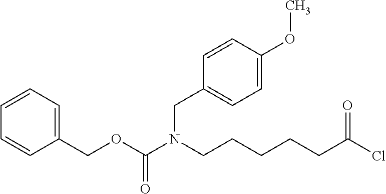

The title compound was prepared analogously to Example 15A from 6-aminocaproic acid.

### Example 17A

### Benzyl (4-chloro-4-oxobutyl)(4-methoxybenzyl)carbamate

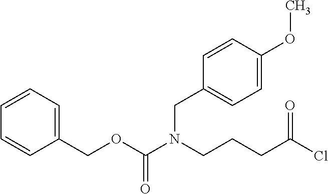

The title compound was prepared analogously to Example 15A from 4-aminobutyric acid.

### Example 18A

### Benzyl butyl(4-chloro-4-oxobutyl)carbamate

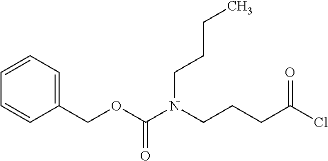

Initially, 4-{[(benzyloxy)carbonyl](butyl)amino}butyric acid was prepared according to a literature procedure [Org. Prep. Proc. Int. 9 (2), 49 (1977)] by lactam opening from N-butylpyrrolidone with subsequent introduction of the Z protective group. Alternatively, the preparation can also be carried out according to [J. Org. Chem. 1985, 50, 1303]. The corresponding acid chloride was then prepared as described in Example 11A.

### Exemplary Embodiments

General Procedure 1 for preparing cesium salts of carboxylic acids or suitably protected amino acid derivatives:

1 mmol of the appropriate carboxylic acid or thiocarboxylic acid is dissolved in a mixture of 10 ml of dioxane and 10 ml of water, and 0.5 mmol of cesium carbonate is added. This is followed by lyophilization.

The Examples 1 to 11 below can, as described in Scheme 1, be prepared by reacting the compound from Example 10A with the cesium salt of the appropriate carboxylic acid or thiocarboxylic acid obtained according to the General Procedure 1.

### Example 1

### 2-[[(5-Chloro-2-thienyl)carbonyl]({(5S)-2-oxo-3-[2-fluoro-4-(3-oxomorpholin-4-yl)phenyl]-1,3-oxazolidin-5-yl}methyl)amino]-2-oxoethyl glycinate hydrochloride

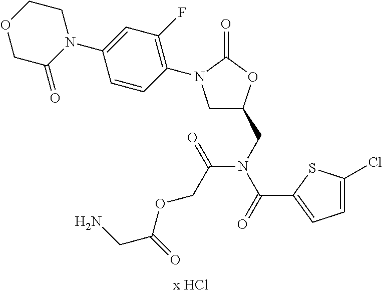

### Example 2

### 2-[[(5-Chloro-2-thienyl)carbonyl]({(5S)-2-oxo-3-[2-fluoro-4-(3-oxomorpholin-4-yl)phenyl]-1,3-oxazolidin-5-yl}methyl)amino]-2-oxoethyl 2-methylalaninate hydrochloride

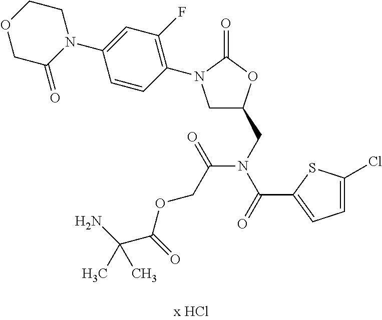

### Example 3

### 2-[[(5-Chloro-2-thienyl)carbonyl]({(5S)-2-oxo-3-[2-fluoro-4-(3-oxomorpholin-4-yl)phenyl]-1,3-oxazolidin-5-yl}methyl)amino]-2-oxoethyl L-valinate hydrochloride

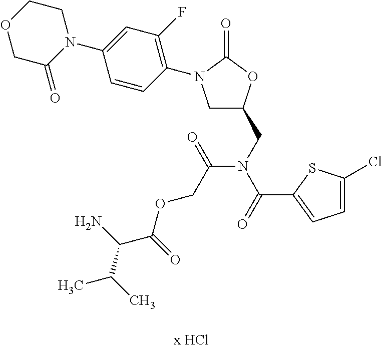

### Example 4

### 2-[[(5-Chloro-2-thienyl)carbonyl]({(5S)-2-oxo-3-[2-fluoro-4-(3-oxomorpholin-4-yl)phenyl]-1,3-oxazolidin-5-yl}methyl)amino]-2-oxoethyl L-prolinate hydrochloride

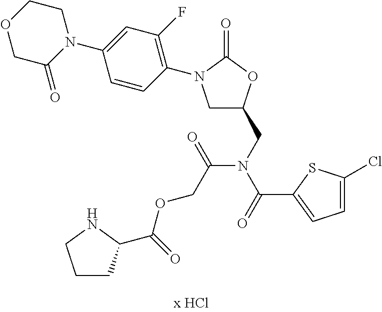

### Example 5

### 2-[[(5-Chloro-2-thienyl)carbonyl]({(5S)-2-oxo-3-[2-fluoro-4-(3-oxomorpholin-4-yl)phenyl]-1,3-oxazolidin-5-yl}methyl)amino]-2-oxoethyl N-methylglycinate hydrochloride

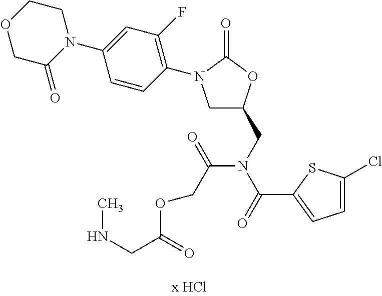

### Example 6

### 2-[[(5-Chloro-2-thienyl)carbonyl]({(5S)-2-oxo-3-[2-fluoro-4-(3-oxomorpholin-4-yl)phenyl]-1,3-oxazolidin-5-yl}methyl)amino]-2-oxoethyl D-valinate hydrochloride

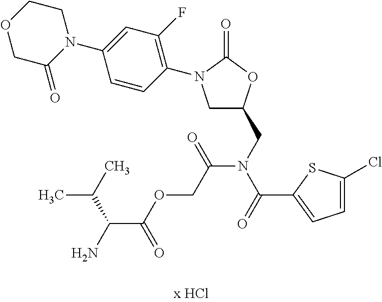

### Example 7

### 2-[[(5-Chloro-2-thienyl)carbonyl]({(5S)-2-oxo-3-[2-fluoro-4-(3-oxomorpholin-4-yl)phenyl]-1,3-oxazolidin-5-yl}methyl)amino]-2-oxoethyl L-lysinate dihydrochloride

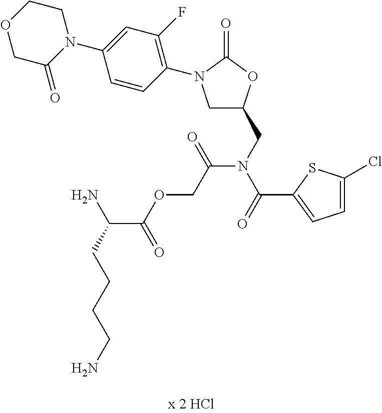

### Example 8

### 2-[[(5-Chloro-2-thienyl)carbonyl]({(5S)-2-oxo-3-[2-fluoro-4-(3-oxomorpholin-4-yl)phenyl]-1,3-oxazolidin-5-yl}methyl)amino]-2-oxoethyl L-histidinate dihydrochloride

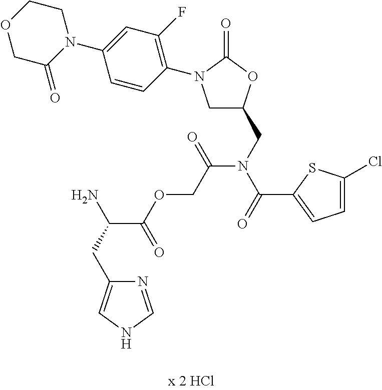

### Example 9

### 2-[[(5-Chloro-2-thienyl)carbonyl]({(5S)-2-oxo-3-[2-fluoro-4-(3-oxomorpholin-4-yl)phenyl]-1,3-oxazolidin-5-yl}methyl)amino]-2-oxoethyl D-histidinate dihydrochloride

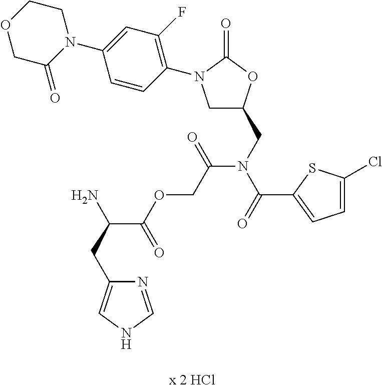

### Example 10

### S-{2-[[(5-Chloro-2-thienyl)carbonyl]({(5S)-2-oxo-3-[2-fluoro-4-(3-oxomorpholin-4-yl)phenyl]-1,3-oxazolidin-5-yl}methyl)amino]-2-oxoethyl}(2S)-2-amino-3-methylbutanethioate hydrobromide

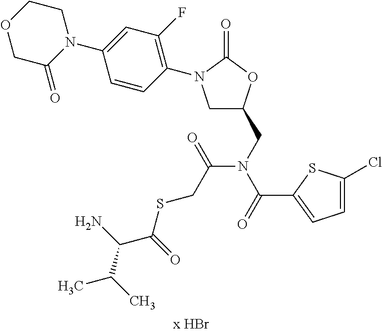

### Example 11

### S-{2-[[(5-Chloro-2-thienyl)carbonyl]({(5S)-2-oxo-3-[2-fluoro-4-(3-oxomorpholin-4-yl)phenyl]-1,3-oxazolidin-5-yl}methyl)amino]-2-oxoethyl}(2S)-2-amino-3-methylbutanethioate hydrochloride

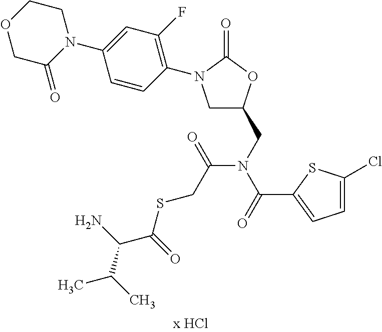

### Example 12

### 5-Chloro-N-[4-(methylamino)butanoyl]-N-({(5S)-2-oxo-3-[2-fluoro-4-(3-oxomorpholin-4-yl)phenyl]-1,3-oxazolidin-5-yl}methyl)thiophene-2-carboxamide hydrobromide

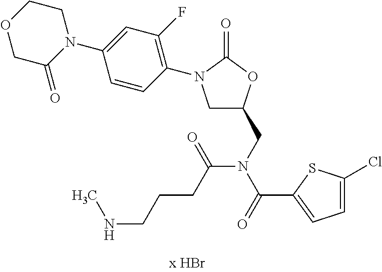

The compound below can be prepared analogously to Example 13 from the appropriate starting compounds. The benzyloxycarbonyl protective group can be removed either directly using hydrogen bromide in glacial acetic acid giving the target compound, or the compound is initially reacted with trifluoroacetic acid and the target compound is isolated after subsequent reaction with hydrogen bromide in glacial acetic acid.

### Example 13

### 5-Chloro-N-[4-(methylamino)butanoyl]-N-({(5S)-2-oxo-3-[2-fluoro-4-(3-oxomorpholin-4-yl)phenyl]-1,3-oxazolidin-5-yl}methyl)thiophene-2-carboxamide hydrochloride

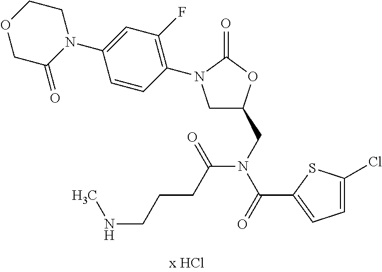

### Step a):

300 mg (0.661 mmol) of the compound (A) were dissolved in 20 ml of DMF, 48 mg (1.98 mmol) of sodium hydride were added and the mixture was stirred at RT for 30 min. 892 mg (3.3 mmol) of the compound from Example 11A dissolved in 2 ml of DMF were then added. The mixture was stirred at RT for a further 15 min, and a few drops of water were then added to the mixture. The mixture was then concentrated and the residue was taken up in 100 ml of dichloromethane. Hydrogen chloride was introduced to saturation into this solution, and the mixture was then allowed to stand overnight. The solution was concentrated and the residue was taken up in 100 ml of ethyl acetate. The mixture was extracted first three times with in each case 50 ml of a 5% strength sodium bicarbonate solution and then once with 50 ml of water. The organic phase was separated off, dried over sodium sulfate and then concentrated. The residue was purified by flash chromatography on silica gel using the mobile phase toluene/ethanol 10:1. The appropriate fractions were combined and concentrated. The residue was twice treated in an ultrasonic bath with 50 ml of ethyl acetate, the solvent was decanted off and the residue was then dried under high vacuum. This gave 130 mg (29%) of the protected compound.

HPLC (Method 7): Rₜ=5.37 min;

LC-MS (Method 9): Rₜ=2.34 min; m/z=687 (M+H)⁺.

### Step b):

127 mg (0.185 mmol) of the Z-protected intermediate obtained above were taken up in 15 ml of trifluoroacetic acid, and the solution was stirred at RT for 3 days. The solution was concentrated and the residue was taken up in 50 ml of water. The mixture was extracted three times with in each case 50 ml of ethyl acetate and concentrated. The residue was dissolved in aqueous hydrochloric acid, which was adjusted to pH 3, and lyophilized. The lyophilizate was once more taken up in aqueous hydrochloric acid, which was adjusted to pH 3, and lyophilized again. What remained were 67 mg (62%) of the title compound.

HPLC (Method 7): Rₜ=4.15 min;

LC-MS (Method 9): Rₜ=0.79 min; m/z=553 (M+H)⁺.

The compounds below can be prepared analogously to Example 13 from the appropriate starting compounds. The trifluoroacetate initially obtained can in each case be used to prepare other salt forms by reaction with the appropriate acid.

### Example 14

### 5-Chloro-N-[5-(methylamino)pentanoyl]-N-({(5S)-2-oxo-3-[2-fluoro-4-(3-oxomorpholin-4-yl)phenyl]-1,3-oxazolidin-5-yl}methyl)thiophene-2-carboxamide hydrobromide

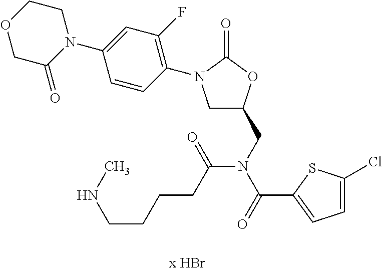

### Example 15

### N-Methylglycyl-N-[(5-chloro-2-thienyl)carbonyl]-N²-methyl-N-({(5S)-2-oxo-3-[2-fluoro-4-(3-oxomorpholin-4-yl)phenyl]-1,3-oxazolidin-5-yl}methyl)glycinamide hydrobromide

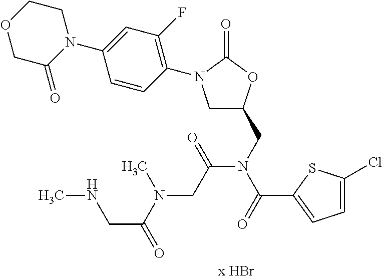

### Example 16

### N-Methyl-β-alanyl-N-[(5-chloro-2-thienyl)carbonyl]-N²-methyl-N-({(5S)-2-oxo-3-[2-fluoro-4-(3-oxomorpholin-4-yl)phenyl]-1,3-oxazolidin-5-yl}methyl)glycinamide hydrobromide

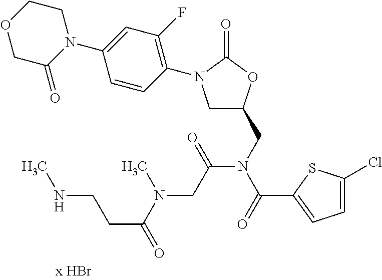

### Example 17

### 5-Chloro-N-[5-(methylamino)pentanoyl]-N-({(5S)-2-oxo-3-[2-fluoro-4-(3-oxomorpholin-4-yl)phenyl]-1,3-oxazolidin-5-yl}methyl)thiophene-2-carboxamide hydrochloride

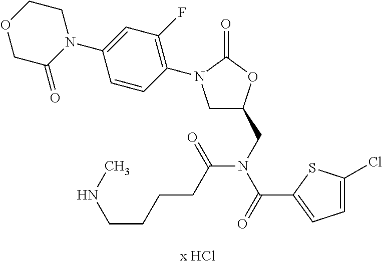

### Example 18

### 5-Chloro-N-[6-(methylamino)hexanoyl]-N-({(5S)-2-oxo-3-[2-fluoro-4-(3-oxomorpholin-4-yl)phenyl]-1,3-oxazolidin-5-yl}methyl)thiophene-2-carboxamide hydrochloride

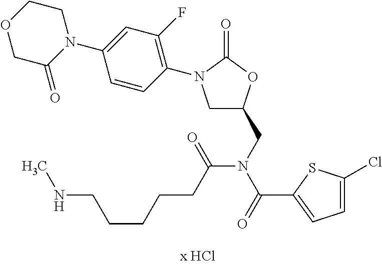

### Example 19

### N-(4-Aminobutanoyl)-5-chloro-N-({(5S)-2-oxo-3-[2-fluoro-4-(3-oxomorpholin-4-yl)phenyl]-1,3-oxazolidin-5-yl}methyl)thiophene-2-carboxamide hydrochloride

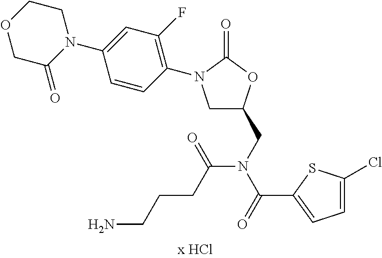

### Step a):

Under an atmosphere of argon, 0.5 g (1.1 mmol) of the compound (A) were dissolved in 27 ml of DMF, 79 mg (3.31 mmol) of sodium hydride were added and the mixture was stirred at RT for 30 min. 4.14 g (11 mmol) of the freshly prepared compound from Example 17A, dissolved in 3 ml of DMF, were then added. Stirring at RT was continued for a further 15 min and 1 ml of methanol was then added to the mixture. The mixture was poured into a 1:1 mixture of 10% strength sodium bicarbonate solution and ethyl acetate. The organic phase was separated off and washed two more times with 10% strength sodium bicarbonate solution. The organic phase was then concentrated, and the residue was stirred at RT with ml of a saturated solution of hydrogen chloride in dichloromethane for 20 h, resulting in the enol ester initially formed being cleaved. The mixture was then concentrated and the residue that remained was purified by flash chromatography on silica gel using the mobile phase toluene/ethyl acetate, the mixing ratio being increased from 1:1 via 1:2 to 1:3. The appropriate fractions were concentrated, giving 124 mg (8% of theory) of the doubly protected compound as a foam.

HPLC (Method 12): Rₜ=2.3 min;

LC-MS (Method 10): Rₜ=2.33 min; m/z=793 (M+H)⁺.

### Step b):

118 mg (0.149 mmol) of the intermediate obtained above were stirred in 6 ml of anhydrous trifluoroacetic acid at RT overnight. The mixture was then concentrated under high vacuum, during which time the temperature was maintained at about 20° C. The residue was taken up in 50 ml of aqueous hydrochloric acid, which was adjusted to pH 3, and 75 ml of dichloromethane were added to the solution. The mixture was shaken, and the aqueous phase was then separated off and concentrated under high vacuum. The residue was purified by preparative HPLC (Method 13). The appropriate fractions were combined, concentrated and then lyophilized from 1N hydrochloric acid. Yield: 59 mg (69% of theory)

HPLC (Method 12): Rₜ=0.98 min;

LC-MS (Method 10): Rₜ=0.98 min; m/z=539 (M+H)⁺.

The compounds below can be prepared analogously to Example 19 from the appropriate starting compounds:

### Example 20

### N-(5-Aminopentanoyl)-5-chloro-N-({(5S)-2-oxo-3-[2-fluoro-4-(3-oxomorpholin-4-yl)phenyl]-1,3-oxazolidin-5-yl}methyl)thiophene-2-carboxamide hydrochloride

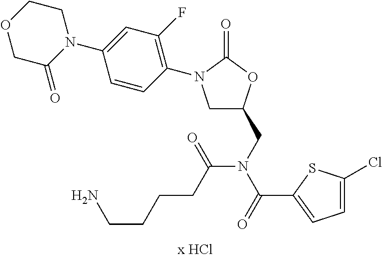

### Example 21

### N-(6-Aminohexanoyl)-5-chloro-N-({(5S)-2-oxo-3-[2-fluoro-4-(3-oxomorpholin-4-yl)phenyl]-1,3-oxazolidin-5-yl}methyl)thiophene-2-carboxamide hydrochloride

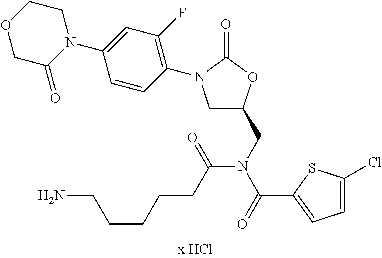

### Example 22

### N-[4-(Butylamino)butanoyl]-5-chloro-N-({(5S)-2-oxo-3-[2-fluoro-4-(3-oxomorpholin-4-yl)phenyl]-1,3-oxazolidin-5-yl}methyl)thiophene-2-carboxamide

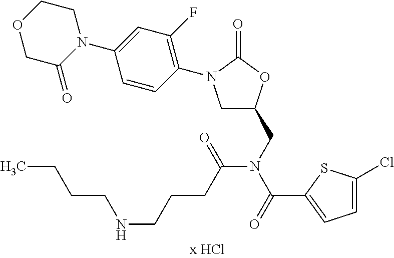

### Step a):

790 mg (1.74 mmol) of the compound (A) were dissolved in 60 ml of DMF, 125 mg (5.22 mmol) of sodium hydride were added and the mixture was stirred at RT for 15 min. 5.43 g (17.4 mmol) of Example 18A, dissolved in 10 ml of DMF, were then added. Stirring at RT was continued for a further 20 min and 10 ml of water were then added to the mixture. The mixture was concentrated and the residue was taken up in 300 ml of ethyl acetate and extracted twice with in each case 50 ml of a 10% strength sodium carbonate solution and once with saturated sodium chloride solution. The ethyl acetate phase was separated off and concentrated. The residue was purified by flash chromatography on silica gel using the mobile phase dichloromethane/acetonitrile, the mixing ratio being increased from 5:1 to 2:1. The appropriate fractions were concentrated. The product that remained was purified by preparative HPLC (Method 1). The fractions which contained the Z-protected intermediate of the title compound were combined and the solvent was removed under reduced pressure. Subsequent drying under high vacuum gave 140 mg (11% of theory) of product.

HPLC (Method 7): Rₜ=6.0 min;

LC-MS (Method 8): Rₜ=4.0 min; m/z=729 (M+H)⁺.

### Step b):

4.7 mg (0.006 mmol) of the protected intermediate were taken up in 5 ml of anhydrous trifluoroacetic acid and the mixture was stirred at RT overnight. The mixture was then concentrated under reduced pressure, with the temperature being maintained at about 20° C. The residue was taken up in 30 ml of a dilute hydrochloric acid which had been adjusted to pH 3 and 10 ml of dichloromethane were added. The phases were separated and the aqueous phase was then extracted once with dichloromethane and subsequently with 5 ml of ethyl acetate. The aqueous phase was concentrated to a volume of about 20 ml under reduced pressure and then lyophilized. The lyophilizate was then once more taken up in hydrochloric acid (pH 3), filtered and lyophilized again. This gave 2.7 mg (66% of theory) of product.

HPLC (Method 7): Rₜ=4.57 min;

LC-MS (Method 8): Rₜ=1.97 min; m/z=595 (M+H)⁺.

The compound below can be prepared analogously to Example 19 from the appropriate starting compounds:

### Example 23

### N-[(2-Aminoethoxy)acetyl]-5-chloro-N-({(5S)-2-oxo-3-[2-fluoro-4-(3-oxomorpholin-4-yl)phenyl]-1,3-oxazolidin-5-yl}methyl)thiophene-2-carboxamide hydrochloride

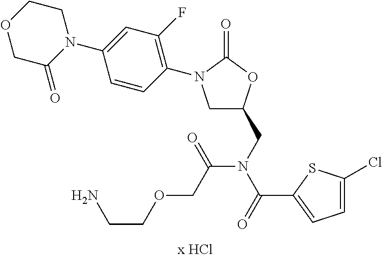

### B. DETERMINATION OF SOLUBILITY, STABILITY AND LIBERATION BEHAVIOR

### a) Determination of the Solubility:

The test substance is suspended in water or dilute hydrochloric acid (pH 4). This suspension is shaken at room temperature for 24 h. After ultracentrifugation at 224 000 g for 30 min, the supernatant is diluted with DMSO and analyzed by HPLC. A two-point calibration plot of the test compound in DMSO is used for quantification.

### HPLC Method:

Agilent 1100 with DAD (G1315A), quat. pump (G1311A), autosampler CTC HTS PAL, degasser (G1322A) and column thermostat (G1316A); column: Zorbax Extend-C18 3.5μ; temperature: 40° C.; mobile phase A: water+5 ml of perchloric acid/liter, mobile phase B: acetonitrile; flow rate: 0.7 ml/min; gradient: 0-0.5 min 98% A, 2% B; ramp 0.5-4.5 min 10% A, 90% B; 4.5-6 min 10% A, 90% B; ramp 6.5-6.7 min 98% A, 2% B; 6.7-7.5 min 98% A, 2% B.

### b) Stability in Buffer at Various pH Values:

0.25 mg of the test substance is weighed into a 2 ml HPLC vial and 0.5 ml of acetonitrile is added. The substance is dissolved by putting the sample vessel in an ultrasonic bath for about 10 seconds. Then 0.5 ml of the respective buffer solution is added, and the sample is again treated in the ultrasonic bath.

### Buffer Solutions Employed:

pH 4.0:1 liter of Millipore water is adjusted to pH 4.0 with 1 N hydrochloric acid;

pH 7.4: 90 g of sodium chloride, 13.61 g of potassium dihydrogen phosphate and 83.35 g of 1 M sodium hydroxide solution are made up to 1 liter with Millipore water and then diluted 1:10.

10 μl portions of the test solution are analyzed by HPLC for their content of unchanged test substance every hour over a period of 24 hours at 37° C. The percentage areas of the appropriate peaks are used for quantification.

### HPLC Method:

Agilent 1100 with DAD (G1314A), binary pump (G1312A), autosampler (G1329A), column oven (G1316A), thermostat (G1330A); column: Kromasil 100 C18, 125 mm×4 mm, 5 μm; column temperature: 30° C.; mobile phase A: water+5 ml of perchloric acid/liter, mobile phase B: acetonitrile.

### Gradient:

0-1.0 min 98% A, 2% B→1.0-13.0 min 50% A, 50% B→13.0-17.0 min 10% A, 90% B→17.0-18.0 min 10% A, 90% B→18.0-19.5 98% A, 2% B→19.5-23.0 min 98% A, 2% B; flow rate: 2.0 ml/min; UV detection: 210 nm.

In solution at pH 4, the compound from Example 22 was stable for more than 16 h.

### c) In Vitro Stability in Rat Plasma and Human Plasma (HPLC Detection):

0.5 mg of substance is dissolved in 1 ml of dimethyl sulfoxide/water 1:1. 500 μl of this sample solution are mixed with 500 μl of rat plasma at 37° C. and shaken. A first sample (10 μl) is immediately taken for HPLC analysis. In the period up to 2 h after the start of incubation, further aliquots are taken after 2, 5, 10, 30, 60 and 90 min, and the contents of the respective test substance and of the active ingredient compound (A) liberated therefrom are determined.

### HPLC-Method:

Agilent 1100 with DAD (G1314A), binary pump (G1312A), autosampler (G1329A), column oven (G1316A), thermostat (G1330A); column: Kromasil 100 C18, 250 mm×4.6 mm, 5 μm; column temperature: 30° C.; mobile phase A: water+5 ml of perchloric acid/liter, mobile phase B: acetonitrile.

### Gradient:

0-3.0 min 69% A, 31% B→3.0-18.0 min 69% A, 31% B→18.0-20.0 min 10% A, 90% B→20.0-21.0 90% A, 10% B→21.0-22.5.0 min 98% A, 2% B→22.5-25.0 min 98% A, 2% B; flow rate: 2.0 ml/min; UV detection: 248 nm.

### Result:

In this test, the compound from Example 22 was degraded both in rat plasma and in human plasma with a half-life of less than 2 min with release of the active ingredient compound (A). In rat plasma, the compounds from Examples 13 and 19 were, within 5 min, converted completely into the active ingredient compound (A).

### d) In Vitro Stability in Rat and Human Plasma (LC/MS-MS Detection):

A defined plasma volume (e.g. 2.0 ml) is warmed to 37° C. in a closed test tube in a waterbath. After the intended temperature is reached, a defined amount of the test substance is added as solution (volume of the solvent not more than 2% of the plasma volume). The plasma is shaken and a first sample (50-100 μl) is immediately taken. Then 4-6 further aliquots are taken in the period up for 2 h after the start of incubation.

Acetonitrile is added to the plasma samples to precipitate proteins. After centrifugation, the test substance and, where appropriate, known cleavage products of the test substance in the supernatant are determined quantitatively with a suitable LC/MS-MS method.

Determinations of stability in heparinized rat or human blood are carried out as described for plasma.

e) i.v. Pharmacokinetics in Wistar Rats:

On the day before administration of the substance, a catheter for obtaining blood is implanted in the jugular vein of the experimental animals (male Wistar rats, body weight 200-250 g) under Isofluran® anesthesia.

On the day of the experiment, a defined dose of the test substance is administered as solution into the tail vein using a Hamilton® glass syringe (bolus administration, duration of administration <10 s). Blood samples (8-12 time points) are taken through the catheter sequentially over the course of 24 h after administration of the substance. Plasma is obtained by centrifuging the samples in heparinized tubes. Acetonitrile is added to a defined plasma volume per time point to precipitate proteins. After centrifugation, test substance and, where appropriate, known cleavage products of the test substance in the supernatant are determined quantitatively using a suitable LC/MS-MS method.

The measured plasma concentrations are used to calculate pharmacokinetic parameters of the test substance and of the active ingredient compound (A) liberated therefrom, such as AUC, Cₘₐₓ, T_{1/2} (half-life) and CL (clearance).

### f) Hepatocyte Assay to Determine the Metabolic Stability:

The metabolic stability of the test compounds in the presence of hepatocytes is determined by incubating the compounds at low concentrations (preferably below 1 μM) and with low cell counts (preferably with 1×10⁶ cells/ml) in order to ensure as far as possible linear kinetic conditions in the experiment. Seven samples of the incubation solution are taken in a fixed time pattern for the LC-MS analysis in order to determine the half-life (i.e. the degradation) of the compound. Various clearance parameters (CL) and Fₘₐₓ values are calculated from this half-life (see below).

The CL and Fₘₐₓ values represent a measure of the phase 1 and phase 2 metabolism of the compound in the hepatocytes. In order to minimize the influence of the organic solvent on the enzymes in the incubation mixtures, its concentration is generally limited to 1% (acetonitrile) or 0.1% (DMSO).

A cell count for hepatocytes in the liver of 1.1×10⁸ cells/g of liver is used for calculation for all species and breeds. CL parameters calculated on the basis of half-lives extending beyond the incubation time (normally 90 minutes) can be regarded only as rough guidelines.

The calculated parameters and their meaning are:
Fₘₐₓ well-stirred [%] maximum possible bioavailability after oral administration
Calculation: (1-CL_{blood} well-stirred/QH)*100
CL_{blood} well-stirred [L/(h*kg)] calculated blood clearance (well stirred model)
Calculation: (QH*CL′_{intrinsic})/(QH+CL′_{intrinsic})
CL′_{intrinsic} [ml/(min*kg)] maximum ability of the liver (of the hepatocytes) to metabolize a compound (on the assumption that the hepatic blood flow is not rate-limiting)
Calculation: CL′_{intrinsic, apparent}*species-specific hepatocyte count [1.1*10⁸/g of liver]*species-specific liver weight [g/kg]
CL′_{intrinsic, apparent} [ml/(min*mg)] normalizes the elimination constant by dividing it by the cell count of hepatocytes employed×(x*10⁶/ml)
Calculation: kₑₗ[1/min]/(cell count [x*10⁶]/incubation volume [ml])
(QH=species-specific hepatic blood flow).

### g) Determination of the Antithrombotic Effect in an Arteriovenous Shunt Model in Rats:

Fasting male rats (strain: HSD CPB:WU) are anesthetized by intraperitoneal administration of a Rompun/Ketavet solution (12 mg/kg/50 mg/kg). Thrombus formation is induced in an arteriovenous shunt based on the method described by P. C. Wong et al. [Thrombosis Research 83 (2), 117-126 (1996)]. For this purpose, the left jugular vein and the right carotid artery are exposed. An 8 cm-long polyethylene catheter (PE60, from Becton-Dickinson) is secured in the artery, followed by a 6 cm-long Tygon tube (R-3606, ID 3.2 mm, from Kronlab) which contains a roughened nylon thread (60×0.26 mm, from Berkley Trilene) made into a double loop to produce a thrombogenic surface. A 2 cm-long polyethylene catheter (PE60, from Becton-Dickinson) is secured in the jugular vein and connected by a 6 cm-long polyethylene catheter (PE160, from Becton-Dickinson) to the Tygon tube. The tubes are filled with physiological saline before the shunt is opened. The extracorporeal circulation is maintained for 15 min. The shunt is then removed and the nylon thread with the thrombus is immediately weighed. The empty weight of the nylon thread has been determined before the start of the experiment. The test substance (as solution in physiological saline adjusted to pH 4 with 0.1 N hydrochloric acid) is administered as bolus injection before attaching the extracorporeal circulation.

### C. EXEMPLARY EMBODIMENTS OF PHARMACEUTICAL COMPOSITIONS

The compounds according to the invention can be converted for example into pharmaceutical preparations in the following way:

i.v. solution:

The compound according to the invention is dissolved at a concentration below the saturation solubility in a physiologically tolerated solvent (e.g. isotonic saline, 5% glucose solution and/or 30% PEG 400 solution, each of which is adjusted to a pH of 3-5). The solution is sterilized by filtration where appropriate and/or dispensed into sterile and pyrogen-free injection containers.

1. A compound of the formula (I) 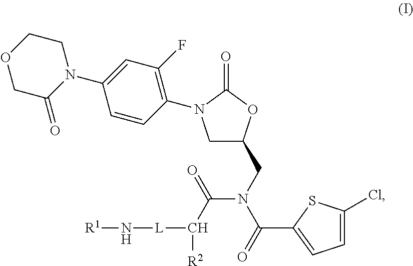
in which
R¹ is hydrogen or (C₁-C₄)-alkyl which may be substituted by hydroxy or (C₁-C₄)-alkoxy,
R² is hydrogen or (C₁-C₄)-alkyl,
and
L is a (C₁-C₄)-alkanediyl group in which one CH₂ group may be replaced by an O atom, or is a group of the formula 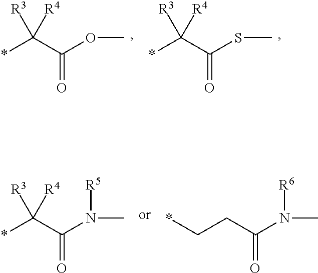
in which
* means the point of linkage to the N atom,
R³ is the side group of a natural α-amino acid or its homologs or isomers,
or
R³ is linked to R¹ and the two together form a (CH₂)₃ or (CH₂)₄ group,
R⁴ is hydrogen or methyl,
R⁵ is (C₁-C₄)-alkyl,
and
R⁶ is hydrogen or (C₁-C₄)-alkyl,
and the salts, solvates and solvates of the salts thereof.

2. The compound of the formula (I) as claimed in claim 1, in which
R¹ is hydrogen or (C₁-C₄)-alkyl,
R² is hydrogen,
and
L is a (C₂-C₄)-alkanediyl group or is a group of the formula 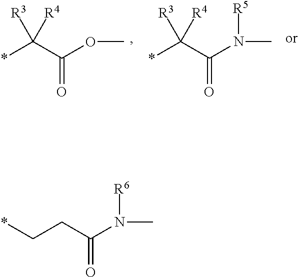
in which
* means the point of linkage to the N atom,
R³ is hydrogen, methyl, propan-2-yl, propan-1-yl, imidazol-4-ylmethyl, hydroxymethyl, 1-hydroxyethyl, carbamoylmethyl, 2-carbamoylethyl, 4-aminobutan-1-yl, 3-aminopropan-1-yl or 3-guanidinopropan-1-yl,
or
R³ is linked to R¹ and the two together form a (CH₂)₃ or (CH₂)₄ group,
R⁴ is hydrogen or methyl,
R⁵ is methyl,
and
R⁶ is hydrogen or methyl,
and the salts, solvates and solvates of the salts thereof.

3. The compound of the formula (I) as claimed in claim 1, in which
R¹ is hydrogen, methyl or n-butyl,
R² is hydrogen,
and
L is a CH₂CH₂ group or is a group of the formula 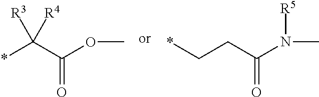
in which
* means the point of linkage to the N atom,
R³ is hydrogen, methyl, propan-2-yl, propan-1-yl, imidazol-4-ylmethyl, hydroxymethyl, 1-hydroxyethyl, carbamoylmethyl, 2-carbamoylethyl, 4-aminobutan-1-yl, 3-aminopropan-1-yl or 3-guanidinopropan-1-yl,
or
R³ is linked to R¹ and the two together form a (CH₂)₃ or (CH₂)₄ group,
R⁴ is hydrogen or methyl,
and
R⁶ is hydrogen or methyl,
and the salts, solvates and solvates of the salts thereof.

4. A process for preparing compounds of the formula (I) as defined in claim 1, characterized in that either
[A] the compound (A) 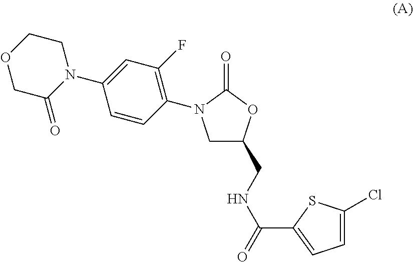
is initially converted in an inert solvent in the presence of a base with a compound of the formula (II) 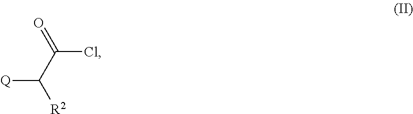
in which R² has the meaning indicated in claims 1 to 3,
and
Q is a leaving group such as, for example, chlorine, bromine or iodine, into a compound of the formula (III) 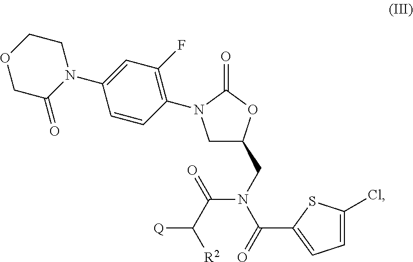
in which Q and R² have the meanings indicated above,
the latter is then reacted in an inert solvent with the cesium salt of an α-amino carboxylic acid or α-amino thiocarboxylic acid of the formula (IV) 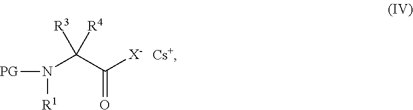
in which R¹, R³ and R⁴ each have the meanings indicated in claim 1, PG is an amino protective group such as, for example, tert-butoxycarbonyl (Boc) or benzyloxycarbonyl (Z),
and
X is O or S,
to give a compound of the formula (V) 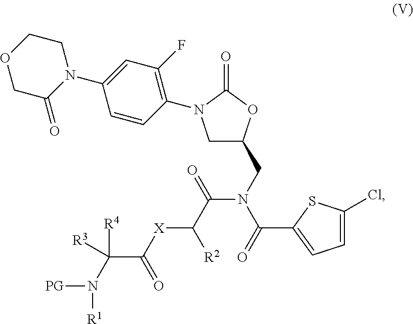
in which R¹, R², R³, R⁴, PG and X each have the meanings indicated above,
and subsequently the protective group PG is removed to result in a compound of the formula (I-A) 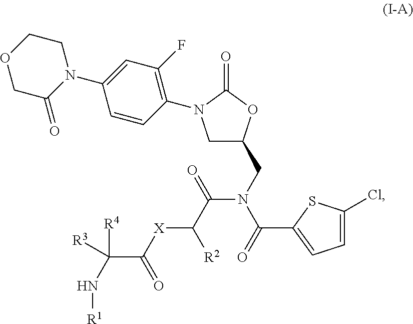
in which R¹, R², R³, R⁴ and X each have the meanings indicated above,
or
[B] compound (A) is reacted in an inert solvent in the presence of a base with a compound of the formula (VI) 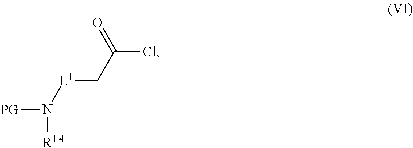
in which PG has the meaning indicated above,
RiA is (C₁-C₄)-alkyl which may be substituted by hydroxy or (C₁-C₄)-alkoxy,
and
L¹ is a (C₁-C₄)-alkanediyl group in which one CH₂ group may be replaced by an O atom, to give a compound of the formula (VII) 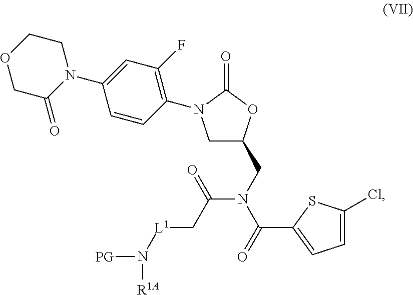
in which R^{1A}, L¹ and PG each have the meanings indicated above,
and subsequently the protective group PG is removed to result in a compound of the formula (I-B) 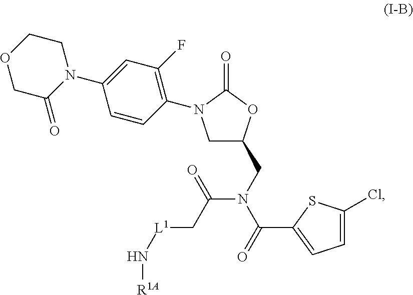
in which R^{1A} and L¹ have the meanings indicated above,
or
[C] the compound (B) 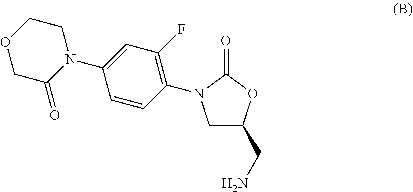
is initially converted into a compound of the formula (VIII) 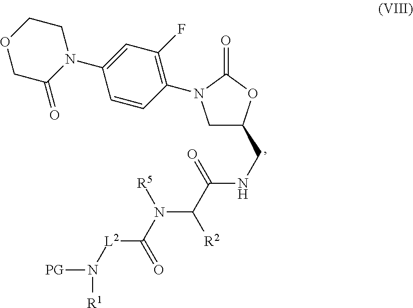
in which PG, R¹, R² and R⁵ each have the meanings indicated in claim 1, and
L² is a (CH₂)₂ or CR³R⁴ group in which R³ and R⁴ each have the meanings indicated in claims 1 to 3,
the latter is then reacted in an inert solvent in the presence of a base with a compound of the formula (IX) 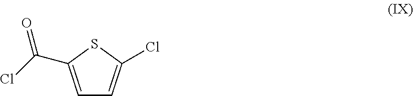
to give a compound of the formula (X) 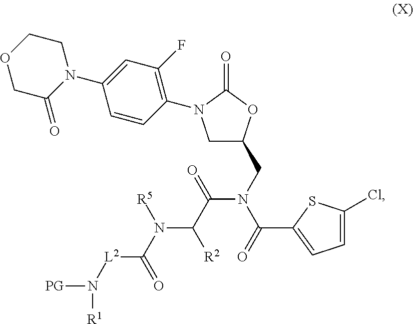
in which PG, L², R¹, R² and R⁵ each have the meanings indicated above,
and subsequently the protective group PG is removed to result in a compound of the formula (I-C) 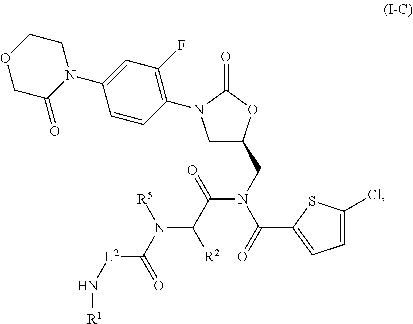
in which L², R¹, R² and R⁵ each have the meanings indicated above,
or
[D] compound (A) is reacted in an inert solvent in the presence of a base with a compound of the formula (XI) 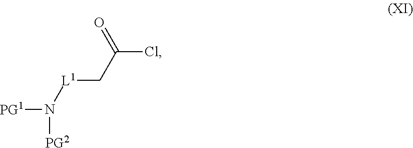
in which
L¹ is a (C₁-C₄)-alkanediyl group in which one CH₂ group may be replaced by an O atom, and
PG¹ and PG² are independently of one another an amino protective group such as, for example, tert-butoxycarbonyl (Boc), benzyloxycarbonyl (Z) or p-methoxybenzyl (PMB) and may be identical or different,
to give a compound of the formula (XII) 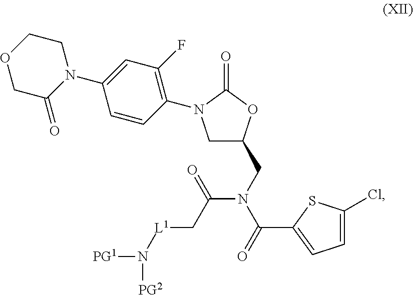
in which L¹, PG¹ and PG² each have the meanings indicated above,
and subsequently the protective groups PG¹ and PG² are removed, simultaneously or sequentially, to result in a compound of the formula (I-D) 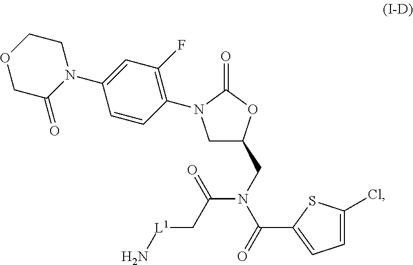
in which L¹ has the meaning indicated above,
and the compounds of the formula (I-A), (I-B), (I-C) and (I-D) resulting in each case are converted where appropriate with the appropriate (i) solvents and/or (ii) acids into the solvates, salts and/or solvates of the salts thereof.

5-6. (canceled)

7. A medicament comprising a compound of the formula (I) as defined in claim 1, where appropriate in combination with an inert, non-toxic, pharmaceutically suitable excipient.

8-9. (canceled)

10. A medicament as claimed in claim 7 for intravenous use.

11. A method for the treatment and/or prophylaxis of thromboembolic disorders in humans and animals using at least one compound of the formula (I) as defined in claim 1.
